SEQUENCE LISTING (260 sequences)

SEQ ID NO: 1 - protein, 253 aa, Artificial
  human prion protein
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 2 - protein, 254 aa, Artificial
  mouse prion protein
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 3 - protein, 253 aa, Artificial; identical to SEQ ID NO: 1
  human prion protein

SEQ ID NO: 4 - protein, 254 aa, Artificial
  Syrian hamster prion protein
MANLSYWLLALFVAMWTDVGLCKKRPKPGGWNTGGSRYPGQGSPGGNRYPPQGGGTWGQPHGGGWGQPHGGGWGQPHGGGWGQPHGGGWGQGGGTHNQWNKPSKPKTNMKHMAGAAAAGAVVGGLGGYMLGSAMSRPMMHFGNDWEDRYYRENMNRYPNQVYYRPVDQYNNQNNFVHDCVNITIKQHTVTTTTKGENFTETDIKIMERVVEQMCTTQYQKESQAYYDGRRSSAVLFSSPPVILLISFLIFLMVG

SEQ ID NO: 5 - protein, 264 aa, Artificial
  bovine prion protein
MVKSHIGSWILVLFVAMWSDVGLCKKRPKPGGGWNTGGSRYPGQGXPGGNRYPPQGGGGWGQPHGGGWGQPHGGGWGQPHGGGWGQPHGGGWGQPHGGGGWGQGGTHGQWNKPSKPKTNMKHVAGAAAAGAVVGGLGGYMLGSAMSRPLIHFGXDYEDRYYRENMHRYPNQVYYRPVDQYSNQNNFVHDCVNITVKEHTVTTTTKGENFTETDIKMMERVVEQMCITQYQRESQAYYQRGASVILFSSPPVILLISFLIFLIVG

SEQ ID NO: 6 - protein, 256 aa, Artificial
  sheep prion protein
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDRYSNQNNFVHDCVNITVKQHTVTTTTKGENFTETDIKIMERVVEQMCITQYQRESQAYYQRGASVILFSSPPVILLISFLIFLIVG

SEQ ID NO: 7 - protein, 254 aa, Artificial; identical to SEQ ID NO: 2
  mouse prion protein

SEQ ID NO: 8 - protein, 256 aa, Artificial
  elk prion protein
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMSRPLIHFGNDYEDRYYRENMYRYPNQVYYRPVDQYNNQNTFVHDCVNITVKQHTVTTTTKGENFTETDIKMMERVVEQMCITQYQRESEAYYQRGASVILFSSPPVILLISFLIFLIVG

SEQ ID NO: 9 - protein, 256 aa, Artificial
  fallow deer prion protein
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMNRPLIHFGNDYEDRYYRENMYRYPNQVYYRPVDQYNNQNTFVHDCVNITVKQHTVTTTTKGENFTETDIKMMERVVEQMCITQYQRESEAYYQRGASVILFSSPPVILLISFLIFLIVG

SEQ ID NO: 10 - protein, 256 aa, Artificial
  mule deer prion protein
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMSRPLIHFGNDYEDRYYRENMYRYPNQVYYRPVDQYNNQNTFVHDCVNITVKQHTVTTTTKGENFTETDIKMMERVVEQMCITQYQRESQAYYQRGASVILFSSPPVILLISFLIFLIVG

SEQ ID NO: 11 - protein, 256 aa, Artificial; identical to SEQ ID NO: 10
  white tailed deer prion protein

SEQ ID NO: 12 - protein, 5 aa, Artificial
  peptide
KKRPK

SEQ ID NO: 13 - protein, 22 aa, Artificial
  peptide
MANLGCWMLVLFVATWSDLGLC

SEQ ID NO: 14 - protein, 14 aa, Artificial
  peptide
GGGQWNKPSKPKTN

SEQ ID NO: 15 - protein, 15 aa, Artificial
  peptide
QWNKPSKPKTNMKHV

SEQ ID NO: 16 - protein, 28 aa, Artificial
  peptide
NQNNXFVHDCVNITXKXHTVTTTTKGEN

SEQ ID NO: 17 - protein, 11 aa, Artificial
  peptide
TTKGENFTETD

SEQ ID NO: 18 - protein, 8 aa, Artificial
  peptide
GENFTETD

SEQ ID NO: 19 - protein, 38 aa, Artificial
  peptide
GENFTETDXKXMERVVEQMCXTQYXESQAYYXGRRXXS

SEQ ID NO: 20 - protein, 63 aa, Artificial
  peptide
NQNNXFVHDCVNITXKXHTVTTTTKGENFTETDXKXMERVVEQMCXTQYXESQAYYXGRRXXS

SEQ ID NO: 21 - protein, 22 aa, Artificial
  peptide
XXLFSSPPVILLISFLIFLXVG

SEQ ID NO: 22 - protein, 36 aa, Artificial
  peptide
GXDXEDRYYRENMXRYPNQVYYRPXDXYXNQNXFVH

SEQ ID NO: 23 - protein, 22 aa, Artificial
  peptide
NXFVHDCVNITXKXHTVTTTTK

SEQ ID NO: 24 - protein, 4 aa, Artificial
  peptide
VYYR

SEQ ID NO: 25 - protein, 13 aa, Artificial
  peptide
RYPNQVYYRPXDX

SEQ ID NO: 26 - protein, 33 aa, Artificial
  peptide
KKRPKPGGGWNTGGSRYPGQGSPGGNRYPPQGG

SEQ ID NO: 27 - protein, 25 aa, Artificial
  peptide
WNTGGSRYPGQGSPGGNRYPPQGGG

SEQ ID NO: 28 - protein, 33 aa, Artificial
  peptide
WNTGGSRYPGQGSPGGNRYPPQGGGXWGQPHGG

SEQ ID NO: 29 - protein, 22 aa, Artificial
  peptide
GGWGQGGGTHSQWNKPSKPKTN

SEQ ID NO: 30 - protein, 16 aa, Artificial
  peptide
GGTHSQWNKPSKPKTN

SEQ ID NO: 31 - protein, 49 aa, Artificial
  peptide
WNTGGSRYPGQGSPGGNRYPPQGGGXWGQPHGGGWGQPHGGGWGQPHGG

SEQ ID NO: 32 - protein, 8 aa, Artificial
  peptide
GQPHGGGW

SEQ ID NO: 33 - protein, 21 aa, Artificial
  peptide
RPIIHFGSDYEDRYYRENMHR

SEQ ID NO: 34 - protein, 21 aa, Artificial
  peptide
RPMIHFGNDWEDRYYRENMYR

SEQ ID NO: 35 - protein, 38 aa, Artificial
  peptide
GGGGCGGGGWGQGGGTHNQWNKPSKPKTNLKHVGGGGC

SEQ ID NO: 36 - protein, 30 aa, Artificial
  peptide
GGGGGGWGQGGGTHNQWNKPSKPKTNLKHV

SEQ ID NO: 37 - protein, 30 aa, Artificial
  peptide
GGWGQGGGTHNQWNKPSKPKTNLKHVGGGG

SEQ ID NO: 38 - protein, 4 aa, Artificial
  peptide
XKHX

SEQ ID NO: 39 - protein, 9 aa, Artificial
  peptide
KPKTNXKHX

SEQ ID NO: 40 - protein, 34 aa, Artificial
  peptide
CGGGGWGQGGGTHNQWNKPSKPKTNLKHVGGGGC

SEQ ID NO: 41 - protein, 25 aa, Artificial
  peptide
SRPIIHFGSDYEDRYYRENMHRYPN

SEQ ID NO: 42 - protein, 23 aa, Artificial
  peptide
PMIHFGNDWEDRYYRENMYRPVD

SEQ ID NO: 43 - protein, 22 aa, Artificial
  peptide
AGAAAAGAVVGGLGGYMLGSAM

SEQ ID NO: 44 - protein, 24 aa, Artificial
  peptide
RPMIHFGNDWEDRYYRENMYRGGG

SEQ ID NO: 45 - protein, 26 aa, Artificial
  peptide
GGGRPMIHFGNDWEDRYYRENMYRGG

SEQ ID NO: 46 - protein, 31 aa, Artificial
  peptide
GGCGGGRPMIHFGNDWEDRYYRENMYRGGGC

SEQ ID NO: 47 - protein, 15 aa, Artificial
  peptide
AGAAAAGAVVGGLGG

SEQ ID NO: 48 - protein, 5 aa, Artificial
  peptide
GGLGG

SEQ ID NO: 49 - protein, 3 aa, Artificial
  peptide
LGS

SEQ ID NO: 50 - protein, 14 aa, Artificial
  peptide
QWNKPSKPKTNGGG

SEQ ID NO: 51 - protein, 25 aa, Artificial
  peptide
QWNKPSKPKTNGGGQWNKPSKPKTN

SEQ ID NO: 52 - protein, 18 aa, Artificial
  peptide
QWNKPSKPKTNLKHVGGG

SEQ ID NO: 53 - protein, 22 aa, Artificial
  peptide
GGWGQGGGTHNQWNKPSKPKTN

SEQ ID NO: 54 - protein, 16 aa, Artificial
  peptide
GGTHNQWNKPSKPKTN

SEQ ID NO: 55 - protein, 25 aa, Artificial
  peptide
GGGAGAAAAGAVVGGLGGYMLGSAM

SEQ ID NO: 56 - protein, 18 aa, Artificial
  peptide
GGGAGAAAAGAVVGGLGG

SEQ ID NO: 57 - protein, 25 aa, Artificial
  peptide
KKKAGAAAAGAVVGGLGGYMLGSAM

SEQ ID NO: 58 - protein, 9 aa, Artificial
  peptide
YMLGSAMXR

SEQ ID NO: 59 - protein, 6 aa, Artificial
  peptide
XRPXXH

SEQ ID NO: 60 - protein, 13 aa, Artificial
  peptide
YMLGSAMXRPXXH

SEQ ID NO: 61 - protein, 17 aa, Artificial
  peptide
YMLGSAMXRPXXHFGXD

SEQ ID NO: 62 - protein, 25 aa, Artificial
  peptide
XEDRYYRENMXRYPNQVYYRPXDXY

SEQ ID NO: 63 - protein, 30 aa, Artificial
  peptide
XEDRYYRENMXRYPNQVYYRPXDXYXNQNX

SEQ ID NO: 64 - protein, 8 aa, Artificial
  peptide
DXYXNQNX

SEQ ID NO: 65 - protein, 18 aa, Artificial
  peptide
KKKAGAAAAGAVVGGLGG

SEQ ID NO: 66 - protein, 24 aa, Artificial
  peptide
GGGKKRPKPGGWNTGGSRYPGQGS

SEQ ID NO: 67 - protein, 16 aa, Artificial
  peptide
GGGKKRPKPGGWNTGG

SEQ ID NO: 68 - protein, 11 aa, Artificial
  peptide
GGGKKRPKPGG

SEQ ID NO: 69 - protein, 23 aa, Artificial
  peptide
PHGGGWGQHGGSWGQPHGGSWGQ

SEQ ID NO: 70 - protein, 16 aa, Artificial
  peptide
PHGGGWGQPHGGSWGQ

SEQ ID NO: 71 - protein, 8 aa, Artificial
  peptide
PHGGGWGQ

SEQ ID NO: 72 - protein, 20 aa, Artificial
  peptide
GGGKKRPKPGGGKKRPKPGG

SEQ ID NO: 73 - protein, 11 aa, Artificial
  peptide
GGGGPKRKGPK

SEQ ID NO: 74 - protein, 16 aa, Artificial
  peptide
GGGWNTGGSRYPGQGS

SEQ ID NO: 75 - protein, 12 aa, Artificial
  peptide
GGGWNKPSKPKT

SEQ ID NO: 76 - protein, 27 aa, Artificial
  peptide
GGGRPMIHFGNDWEDRYYRENMYRGGC

SEQ ID NO: 77 - protein, 18 aa, Artificial
  peptide
QWNKPSKPKTNLKHVGGG

SEQ ID NO: 78 - protein, 25 aa, Artificial
  peptide
GGGAGAAAAGAVVGGLGGYMLGSAM

SEQ ID NO: 79 - protein, 10 aa, Artificial
  peptide
GGGNKPSKPK

SEQ ID NO: 80 - protein, 9 aa, Artificial
  peptide
GGGKPSKPK

SEQ ID NO: 81 - protein, 23 aa, Artificial
  peptide
GGGKKRPKPGGGQWNKPSKPKTN

SEQ ID NO: 82 - protein, 28 aa, Artificial
  peptide
KKKAGAAAAGAVVGGLGGYMLGSAMDDD

SEQ ID NO: 83 - protein, 25 aa, Artificial
  peptide
DDDAGAAAAGAVVGGLGGYMLGSAM

SEQ ID NO: 84 - protein, 28 aa, Artificial
  peptide
KKKAGAAAAGAVVGGLGGYMLGSAMKKK

SEQ ID NO: 85 - protein, 11 aa, Artificial
  peptide
GGGKKKKKKKK

SEQ ID NO: 86 - protein, 28 aa, Artificial
  peptide
DDDAGAAAAGAVVGGLGGYMLGSAMDDD

SEQ ID NO: 87 - protein, 14 aa, Artificial
  peptide
GGGNNKQSPWPTKK

SEQ ID NO: 88 - protein, 23 aa, Artificial
  peptide
DKDKGGVGALAGAAVAAGGDKDK

SEQ ID NO: 89 - protein, 14 aa, Artificial
  peptide
GGGQANKPSKPKTN

SEQ ID NO: 90 - protein, 14 aa, Artificial
  peptide
GGGQWNKASKPKTN

SEQ ID NO: 91 - protein, 14 aa, Artificial
  peptide
GGGQWNKPSKAKTN

SEQ ID NO: 92 - protein, 14 aa, Artificial
  peptide
GGGQWNAPSKPKTN

SEQ ID NO: 93 - protein, 14 aa, Artificial
  peptide
GGGQWNKPSAPKTN

SEQ ID NO: 94 - protein, 14 aa, Artificial
  peptide
GGGQWNKPSKPATN

SEQ ID NO: 95 - protein, 14 aa, Artificial
  peptide
GGGQWNKASKAKTN

SEQ ID NO: 96 - protein, 11 aa, Artificial
  peptide
GGGKKRAKPGG

SEQ ID NO: 97 - protein, 11 aa, Artificial
  peptide
GGGKKRPKAGG

SEQ ID NO: 98 - protein, 11 aa, Artificial
  peptide
GGGKKRAKAGG

SEQ ID NO: 99 - protein, 14 aa, Artificial
  peptide
GGGQWNKASKPKTN

SEQ ID NO: 100 - protein, 14 aa, Artificial
  peptide
GGGQWAKPSKPKTN

SEQ ID NO: 101 - protein, 14 aa, Artificial
  peptide
GGGQWNKPAKPKTN

SEQ ID NO: 102 - protein, 14 aa, Artificial
  peptide
GGGQWNKPSKPKAN

SEQ ID NO: 103 - protein, 14 aa, Artificial
  peptide
GGGQWNKPSKPKTA

SEQ ID NO: 104 - protein, 11 aa, Artificial
  peptide
GGGAKRPKPGG

SEQ ID NO: 105 - protein, 11 aa, Artificial
  peptide
GGGKARPKPGG

SEQ ID NO: 106 - protein, 11 aa, Artificial
  peptide
GGGKKAPKPGG

SEQ ID NO: 107 - protein, 11 aa, Artificial
  peptide
GGGKKRPAPGG

SEQ ID NO: 108 - protein, 11 aa, Artificial
  peptide
GGGKKAPKAGG

SEQ ID NO: 109 - protein, 14 aa, Artificial
  peptide
GGGQWNKPSKGKTN

SEQ ID NO: 110 - protein, 14 aa, Artificial
  peptide
GGGQWNKPSKGKTN

SEQ ID NO: 111 - protein, 14 aa, Artificial
  peptide
GGGQWNKPSKGKTN

SEQ ID NO: 112 - protein, 14 aa, Artificial
  peptide
GGGQWNKPSKGKTN

SEQ ID NO: 113 - protein, 14 aa, Artificial
  peptide
GGGQWNKPSKGKTN

SEQ ID NO: 114 - protein, 14 aa, Artificial
  peptide
GGGQWNKPSKGKTN

SEQ ID NO: 115 - protein, 14 aa, Artificial
  peptide
GGGQWNKGSKPKTN

SEQ ID NO: 116 - protein, 14 aa, Artificial
  peptide
GGGQWNKGSKPKTN

SEQ ID NO: 117 - protein, 14 aa, Artificial
  peptide
GGGQWNKGSKPKTN

SEQ ID NO: 118 - protein, 14 aa, Artificial
  peptide
GGGQWNKGSKPKTN

SEQ ID NO: 119 - protein, 11 aa, Artificial
  peptide
GGGKKRPKGGG

SEQ ID NO: 120 - protein, 11 aa, Artificial
  peptide
GGGKKRPKGGG

SEQ ID NO: 121 - protein, 11 aa, Artificial
  peptide
GGGKKRPKGGG

SEQ ID NO: 122 - protein, 11 aa, Artificial
  peptide
GGGKKRPKGGG

SEQ ID NO: 123 - protein, 16 aa, Artificial
  peptide
GGGKKRPKGGGWNTGG

SEQ ID NO: 124 - protein, 16 aa, Artificial
  peptide
GGGKKRPKGGGWNTGG

SEQ ID NO: 125 - protein, 16 aa, Artificial
  peptide
GGGKKRPKGGGWNTGG

SEQ ID NO: 126 - protein, 16 aa, Artificial
  peptide
GGGKKRPKGGGWNTGG

SEQ ID NO: 127 - protein, 17 aa, Artificial
  peptide
GGGKKRPKPGGGWNTGG

SEQ ID NO: 128 - protein, 39 aa, Artificial
  peptide
QWNKPSKPKTNGGGQWNKPSKPKTNGGGQWNKPSKPKTN

SEQ ID NO: 129 - protein, 14 aa, Artificial
  peptide
GGGQWNKGSKGKTN

SEQ ID NO: 130 - protein, 14 aa, Artificial
  peptide
GGGQWNKGSKGKTN

SEQ ID NO: 131 - protein, 14 aa, Artificial
  peptide
GGGQWNKGSKGKTN

SEQ ID NO: 132 - protein, 14 aa, Artificial
  peptide
GGGQWNKGSKGKTN

SEQ ID NO: 133 - protein, 23 aa, Artificial
  peptide
QWNKPSKPKTNQWNKPSKPKTNK

SEQ ID NO: 134 - protein, 68 aa, Artificial
  peptide
GGGKKRPKPGGWNTGGGGGGKKRPKPGGWNTGGGGGGKKRPKPGGWNTGGGGGGKKRPKPGGWNTGGG

SEQ ID NO: 135 - protein, 136 aa, Artificial
  peptide
GGGKKRPKPGGWNTGGGGGGKKRPKPGGWNTGGGGGGKKRPKPGGWNTGGGGGGKKRPKPGGWNTGGGGGGKKRPKPGGWNTGGGGGGKKRPKPGGWNTGGGGGGKKRPKPGGWNTGGGGGGKKRPKPGGWNTGGG

SEQ ID NO: 136 - protein, 18 aa, Artificial
  peptide
KKKAGAAAAGAVVGGLGG

SEQ ID NO: 137 - protein, 19 aa, Artificial
  peptide
DLGLCKKRPKPGGXWNTGG

SEQ ID NO: 138 - protein, 18 aa, Artificial
  peptide
DLGLCKKRPKPGGXWNTG

SEQ ID NO: 139 - protein, 17 aa, Artificial
  peptide
DLGLCKKRPKPGGXWNT

SEQ ID NO: 140 - protein, 16 aa, Artificial
  peptide
DLGLCKKRPKPGGXWN

SEQ ID NO: 141 - protein, 15 aa, Artificial
  peptide
DLGLCKKRPKPGGXW

SEQ ID NO: 142 - protein, 14 aa, Artificial
  peptide
DLGLCKKRPKPGGX

SEQ ID NO: 143 - protein, 17 aa, Artificial
  peptide
LGLCKKRPKPGGXWNTG

SEQ ID NO: 144 - protein, 16 aa, Artificial
  peptide
LGLCKKRPKPGGXWNT

SEQ ID NO: 145 - protein, 15 aa, Artificial
  peptide
LGLCKKRPKPGGXWN

SEQ ID NO: 146 - protein, 14 aa, Artificial
  peptide
LGLCKKRPKPGGXW

SEQ ID NO: 147 - protein, 13 aa, Artificial
  peptide
LGLCKKRPKPGGX

SEQ ID NO: 148 - protein, 17 aa, Artificial
  peptide
GLCKKRPKPGGXWNTGG

SEQ ID NO: 149 - protein, 16 aa, Artificial
  peptide
GLCKKRPKPGGXWNTG

SEQ ID NO: 150 - protein, 15 aa, Artificial
  peptide
GLCKKRPKPGGXWNT

SEQ ID NO: 151 - protein, 14 aa, Artificial
  peptide
GLCKKRPKPGGXWN

SEQ ID NO: 152 - protein, 13 aa, Artificial
  peptide
GLCKKRPKPGGXW

SEQ ID NO: 153 - protein, 12 aa, Artificial
  peptide
GLCKKRPKPGGX

SEQ ID NO: 154 - protein, 16 aa, Artificial
  peptide
LCKKRPKPGGXWNTGG

SEQ ID NO: 155 - protein, 15 aa, Artificial
  peptide
LCKKRPKPGGXWNTG

SEQ ID NO: 156 - protein, 14 aa, Artificial
  peptide
LCKKRPKPGGXWNT

SEQ ID NO: 157 - protein, 13 aa, Artificial
  peptide
LCKKRPKPGGXWN

SEQ ID NO: 158 - protein, 12 aa, Artificial
  peptide
LCKKRPKPGGXW

SEQ ID NO: 159 - protein, 11 aa, Artificial
  peptide
LCKKRPKPGGX

SEQ ID NO: 160 - protein, 15 aa, Artificial
  peptide
CKKRPKPGGXWNTGG

SEQ ID NO: 161 - protein, 14 aa, Artificial
  peptide
CKKRPKPGGXWNTG

SEQ ID NO: 162 - protein, 13 aa, Artificial
  peptide
CKKRPKPGGXWNT

SEQ ID NO: 163 - protein, 12 aa, Artificial
  peptide
CKKRPKPGGXWN

SEQ ID NO: 164 - protein, 11 aa, Artificial
  peptide
CKKRPKPGGXW

SEQ ID NO: 165 - protein, 10 aa, Artificial
  peptide
CKKRPKPGGX

SEQ ID NO: 166 - protein, 14 aa, Artificial
  peptide
KKRPKPGGXWNTGG

SEQ ID NO: 167 - protein, 13 aa, Artificial
  peptide
KKRPKPGGXWNTG

SEQ ID NO: 168 - protein, 12 aa, Artificial
  peptide
KKRPKPGGXWNT

SEQ ID NO: 169 - protein, 11 aa, Artificial
  peptide
KKRPKPGGXWN

SEQ ID NO: 170 - protein, 10 aa, Artificial
  peptide
KKRPKPGGXW

SEQ ID NO: 171 - protein, 9 aa, Artificial
  peptide
KKRPKPGGX

SEQ ID NO: 172 - protein, 19 aa, Artificial
  peptide
DVGLCKKRPKPGGXWNTGG

SEQ ID NO: 173 - protein, 18 aa, Artificial
  peptide
DVGLCKKRPKPGGXWNTG

SEQ ID NO: 174 - protein, 17 aa, Artificial
  peptide
DVGLCKKRPKPGGXWNT

SEQ ID NO: 175 - protein, 16 aa, Artificial
  peptide
DVGLCKKRPKPGGXWN

SEQ ID NO: 176 - protein, 15 aa, Artificial
  peptide
DVGLCKKRPKPGGXW

SEQ ID NO: 177 - protein, 14 aa, Artificial
  peptide
DVGLCKKRPKPGGX

SEQ ID NO: 178 - protein, 17 aa, Artificial
  peptide
VGLCKKRPKPGGXWNTG

SEQ ID NO: 179 - protein, 16 aa, Artificial
  peptide
VGLCKKRPKPGGXWNT

SEQ ID NO: 180 - protein, 15 aa, Artificial
  peptide
VGLCKKRPKPGGXWN

SEQ ID NO: 181 - protein, 14 aa, Artificial
  peptide
VGLCKKRPKPGGXW

SEQ ID NO: 182 - protein, 13 aa, Artificial
  peptide
VGLCKKRPKPGGX

SEQ ID NO: 183 - protein, 18 aa, Artificial
  peptide
THSQWNKPSKPKTNMKHM

SEQ ID NO: 184 - protein, 17 aa, Artificial
  peptide
THSQWNKPSKPKTNMKH

SEQ ID NO: 185 - protein, 16 aa, Artificial
  peptide
THSQWNKPSKPKTNMK

SEQ ID NO: 186 - protein, 15 aa, Artificial
  peptide
THSQWNKPSKPKTNM

SEQ ID NO: 187 - protein, 14 aa, Artificial
  peptide
THSQWNKPSKPKTN

SEQ ID NO: 188 - protein, 17 aa, Artificial
  peptide
HSQWNKPSKPKTNMKHM

SEQ ID NO: 189 - protein, 16 aa, Artificial
  peptide
HSQWNKPSKPKTNMKH

SEQ ID NO: 190 - protein, 15 aa, Artificial
  peptide
HSQWNKPSKPKTNMK

SEQ ID NO: 191 - protein, 14 aa, Artificial
  peptide
HSQWNKPSKPKTNM

SEQ ID NO: 192 - protein, 13 aa, Artificial
  peptide
HSQWNKPSKPKTN

SEQ ID NO: 193 - protein, 16 aa, Artificial
  peptide
SQWNKPSKPKTNMKHM

SEQ ID NO: 194 - protein, 15 aa, Artificial
  peptide
SQWNKPSKPKTNMKH

SEQ ID NO: 195 - protein, 14 aa, Artificial
  peptide
SQWNKPSKPKTNMK

SEQ ID NO: 196 - protein, 13 aa, Artificial
  peptide
SQWNKPSKPKTNM

SEQ ID NO: 197 - protein, 12 aa, Artificial
  peptide
SQWNKPSKPKTN

SEQ ID NO: 198 - protein, 15 aa, Artificial
  peptide
QWNKPSKPKTNMKHM

SEQ ID NO: 199 - protein, 14 aa, Artificial
  peptide
QWNKPSKPKTNMKH

SEQ ID NO: 200 - protein, 13 aa, Artificial
  peptide
QWNKPSKPKTNMK

SEQ ID NO: 201 - protein, 12 aa, Artificial
  peptide
QWNKPSKPKTNM

SEQ ID NO: 202 - protein, 18 aa, Artificial
  peptide
THSQWNKPSKPKTNMKHV

SEQ ID NO: 203 - protein, 17 aa, Artificial
  peptide
HSQWNKPSKPKTNMKHV

SEQ ID NO: 204 - protein, 16 aa, Artificial
  peptide
SQWNKPSKPKTNMKHV

SEQ ID NO: 205 - protein, 15 aa, Artificial
  peptide
QWNKPSKPKTNMKHV

SEQ ID NO: 206 - protein, 18 aa, Artificial
  peptide
THGQWNKPSKPKTNMKHM

SEQ ID NO: 207 - protein, 17 aa, Artificial
  peptide
THGQWNKPSKPKTNMKH

SEQ ID NO: 208 - protein, 16 aa, Artificial
  peptide
THGQWNKPSKPKTNMK

SEQ ID NO: 209 - protein, 15 aa, Artificial
  peptide
THGQWNKPSKPKTNM

SEQ ID NO: 210 - protein, 14 aa, Artificial
  peptide
THGQWNKPSKPKTN

SEQ ID NO: 211 - protein, 17 aa, Artificial
  peptide
HGQWNKPSKPKTNMKHM

SEQ ID NO: 212 - protein, 16 aa, Artificial
  peptide
HGQWNKPSKPKTNMKH

SEQ ID NO: 213 - protein, 15 aa, Artificial
  peptide
HGQWNKPSKPKTNMK

SEQ ID NO: 214 - protein, 14 aa, Artificial
  peptide
HGQWNKPSKPKTNM

SEQ ID NO: 215 - protein, 13 aa, Artificial
  peptide
HGQWNKPSKPKTN

SEQ ID NO: 216 - protein, 16 aa, Artificial
  peptide
GQWNKPSKPKTNMKHM

SEQ ID NO: 217 - protein, 15 aa, Artificial
  peptide
GQWNKPSKPKTNMKH

SEQ ID NO: 218 - protein, 14 aa, Artificial
  peptide
GQWNKPSKPKTNMK

SEQ ID NO: 219 - protein, 13 aa, Artificial
  peptide
GQWNKPSKPKTNM

SEQ ID NO: 220 - protein, 12 aa, Artificial
  peptide
GQWNKPSKPKTN

SEQ ID NO: 221 - protein, 18 aa, Artificial
  peptide
THGQWNKPSKPKTNMKHV

SEQ ID NO: 222 - protein, 17 aa, Artificial
  peptide
HGQWNKPSKPKTNMKHV

SEQ ID NO: 223 - protein, 16 aa, Artificial
  peptide
GQWNKPSKPKTNMKHV

SEQ ID NO: 224 - protein, 18 aa, Artificial
  peptide
THNQWNKPSKPKTNMKHM

SEQ ID NO: 225 - protein, 17 aa, Artificial
  peptide
THNQWNKPSKPKTNMKH

SEQ ID NO: 226 - protein, 16 aa, Artificial
  peptide
THNQWNKPSKPKTNMK

SEQ ID NO: 227 - protein, 15 aa, Artificial
  peptide
THNQWNKPSKPKTNM

SEQ ID NO: 228 - protein, 14 aa, Artificial
  peptide
THNQWNKPSKPKTN

SEQ ID NO: 229 - protein, 17 aa, Artificial
  peptide
HNQWNKPSKPKTNMKHM

SEQ ID NO: 230 - protein, 16 aa, Artificial
  peptide
HNQWNKPSKPKTNMKH

SEQ ID NO: 231 - protein, 15 aa, Artificial
  peptide
HNQWNKPSKPKTNMK

SEQ ID NO: 232 - protein, 14 aa, Artificial
  peptide
HNQWNKPSKPKTNM

SEQ ID NO: 233 - protein, 13 aa, Artificial
  peptide
HNQWNKPSKPKTN

SEQ ID NO: 234 - protein, 16 aa, Artificial
  peptide
NQWNKPSKPKTNMKHM

SEQ ID NO: 235 - protein, 15 aa, Artificial
  peptide
NQWNKPSKPKTNMKH

SEQ ID NO: 236 - protein, 14 aa, Artificial
  peptide
NQWNKPSKPKTNMK

SEQ ID NO: 237 - protein, 13 aa, Artificial
  peptide
NQWNKPSKPKTNM

SEQ ID NO: 238 - protein, 12 aa, Artificial
  peptide
NQWNKPSKPKTN

SEQ ID NO: 239 - protein, 18 aa, Artificial
  peptide
THNQWNKPSKPKTNMKHV

SEQ ID NO: 240 - protein, 17 aa, Artificial
  peptide
HNQWNKPSKPKTNMKHV

SEQ ID NO: 241 - protein, 16 aa, Artificial
  peptide
NQWNKPSKPKTNMKHV

SEQ ID NO: 242 - protein, 24 aa, Artificial
  peptide
PHGGGWGQPHGGGWGQPHGGGWGQ

SEQ ID NO: 243 - protein, 26 aa, Artificial
  peptide
GGWGQGGGTHSQWNKPSKPKTNMKHM

SEQ ID NO: 244 - protein, 33 aa, Artificial
  peptide
QWNKPSKPKTNMKHMGGGQWNKPSKPKTNMKHM

SEQ ID NO: 245 - protein, 30 aa, Artificial
  peptide
GGWGQGGGTHXQWNKPSKPKTNXKHXGGGG

SEQ ID NO: 246 - protein, 23 aa, Artificial
  peptide
PHGGGWGQHGXSWGQPHGGXWGQ

SEQ ID NO: 247 - protein, 18 aa, Artificial
  peptide
QWNKPSKPKTNXKHXGGG

SEQ ID NO: 248 - protein, 14 aa, Artificial
  peptide
GGGAWNKPSKPKTN

SEQ ID NO: 249 - protein, 16 aa, Artificial
  peptide
GGGKKRGKPGGWNTGG

SEQ ID NO: 250 - protein, 16 aa, Artificial
  peptide
GGGKKRGKPGGWNTGG

SEQ ID NO: 251 - protein, 16 aa, Artificial
  peptide
GGGKKRGKPGGWNTGG

SEQ ID NO: 252 - protein, 16 aa, Artificial
  peptide
GGGKKRGKPGGWNTGG

SEQ ID NO: 253 - protein, 11 aa, Artificial
  peptide
GGGKKRGKPGG

SEQ ID NO: 254 - protein, 11 aa, Artificial
  peptide
GGGKKRGKPGG

SEQ ID NO: 255 - protein, 11 aa, Artificial
  peptide
GGGKKRGKPGG

SEQ ID NO: 256 - protein, 11 aa, Artificial
  peptide
GGGKKRGKPGG

SEQ ID NO: 257 - protein, 14 aa, Artificial
  peptide
GGGQWNKGSKGKTN

SEQ ID NO: 258 - protein, 14 aa, Artificial
  peptide
GGGQWNKGSKGKTN

SEQ ID NO: 259 - protein, 68 aa, Artificial
  peptide
GGGQWNKPSKPKTNGGGGGGQWNKPSKPKTNGGGGGGQWNKPSKPKTNGGGGGGQWNKPSKPKTNGGG

SEQ ID NO: 260 - protein, 136 aa, Artificial; identical to SEQ ID NO: 135
  peptide

(19) United States
(12) Patent Application Publication
(10) Pub. No.: US 2009/0099343 A1
(43) Pub. Date: Apr. 16, 2009

(54) Isolation of pathogenic prions

(76) Inventors: David Peretz, El Cerrito, CA (US); Melissa Michelitsch, Berkeley, CA (US); Celine Hu, Tiburon, CA (US); David Chien, Alamo, CA (US); John Hall, Emeryville, CA (US); Xuemei Wang, Castro Valley, CA (US); Man Gao, San Ramon, CA (US)

Correspondence Address: NOVARTIS VACCINES AND DIAGNOSTICS INC., INTELLECTUAL PROPERTY R338, P.O. BOX 8097, Emeryville, CA, 94662-8097, US

(21) Appl. No.: 11/795,165

(22) PCT Filed: Jan. 13, 2006
(86) PCT No.: PCT/US06/01090

§ 371 Date: Oct. 17, 2008

Related U.S. Application Data
(60) Provisional application No. 60/644,185, filed on Jan. 13, 2005.

(51) Int. Cl. C07K 1/14

(52) U.S. Cl. 530/412; 530/418

(57) ABSTRACT

Peptide reagents that interact preferentially with the PrP^{sc} form of the prion protein are described. Methods of using the reagents for isolation and purification of the PrP^{sc} isoform are described.

### FIELD OF THE INVENTION

The invention relates to use of peptide reagents that interact with prion proteins for the isolation of pathogenic prion proteins.

### BACKGROUND

Protein conformational diseases include a variety of unrelated diseases, including transmissible spongiform encephalopathies, arising from aberrant conformational transition of a protein (a conformational disease protein) which in turn leads to self-association of the aberrant protein forms, with consequent tissue deposition and damage. These diseases also share striking similarities in clinical presentations, typically a rapid progression from diagnosis to death following varying lengths of incubation.

One group of conformational diseases are termed “prion diseases” or “transmissible spongiform encephalopathies (TSEs).” In humans these diseases include Creutzfeldt-Jakob disease (CJD), Gerstmann-Straussler-Scheinker syndrome (GSS), Fatal Familial Insomnia, and Kuru (see, e.g., Harrison's Principles of Internal Medicine, Isselbacher et al., eds., McGraw-Hill, Inc. New York, (1994); Medori et al. (1992) N. Engl. J. Med. 326: 444-9.). In animals the TSE's include sheep scrapie, bovine spongiform encephalopathy (BSE), transmissible mink encephalopathy, and chronic wasting disease of captive mule deer and elk (Gajdusek, (1990) Subacute Spongiform Encephalopathies: Transmissible Cerebral Amyloidoses Caused by Unconventional Viruses. Pp. 2289-2324 In: Virology, Fields, ed. New York: Raven Press, Ltd.). Transmissible spongiform encephalopathies are characterized by the same hallmarks: the presence of the abnormal (beta-rich, proteinase K resistant) conformation of the prion protein that transmits disease when experimentally inoculated into laboratory animals including primates, rodents, and transgenic mice.

Recently, the rapid spread of bovine spongiform encephalopathy and its correlation with elevated occurrence of spongiform encephalopathies in humans has lead to a significant increase of interest in the detection of transmissible spongiform encephalopathies in non-human mammals. The tragic consequences of accidental transmission of these diseases (see, e.g., Gajdusek, Infectious Amyloids, and Prusiner Prions In Fields Virology. Fields, et al., eds. Lippincott-Ravin, Pub. Philadelphia (1996); Brown et al. (1992) Lancet, 340: 24-27), decontamination difficulties (Asher et al. (1986) pages 59-71 In: Laboratory Safety: Principles and Practices, Miller ed. Am. Soc. Microb.), and recent concern about bovine spongiform encephalopathy (British Med. J. (1995) 311: 1415-1421) underlie the urgency of having both a diagnostic test that would identify humans and animals with transmissible spongiform encephalopathies and therapies for infected subjects.

Prions are the infectious pathogen that causes spongiform encephalopathies (prion diseases). Prions differ significantly from bacteria, viruses and viroids. The dominating hypothesis is that, unlike all other infectious pathogens, infection is caused by an abnormal conformation of the prion protein, which acts as a template and converts normal prion conformations into abnormal conformations. A prion protein was first characterized in the early 1980s. (See, e.g., Bolton, McKinley et al. (1982) Science 218:1309-1311; Prusiner, Bolton et al. (1982) Biochemistry 21:6942-6950; McKinley, Bolton et al. (1983) Cell 35:57-62). Complete prion protein-encoding genes have since been cloned, sequenced and expressed in transgenic animals. See, e.g., Basler, Oesch et al. (1986) Cell 46:417-428.

The key characteristic of prion diseases is the formation of an abnormally shaped protein (PrP^{Sc}), also referred to as a scrapie protein, from the normal (cellular or nonpathogenic) form of prion protein (PrP^{C}). See, e.g., Zhang et al. (1997) Biochem. 36(12):3543-3553; Cohen & Prusiner (1998) Ann Rev. Biochem. 67:793-819; Pan et al. (1993) Proc Nat'l Acad Sci USA 90:10962-10966; Safar et al. (1993) J Biol Chem 268:20276-20284. Optical spectroscopy and crystallography studies have revealed that disease-related forms of prions are substantially enriched in beta-sheet structure as compared to the predominantly alpha-helical folded non-disease forms. See, e.g., Wille et al. (2001) Proc. Nat'l Acad. Sci. USA 99:3563-3568; Peretz et al. (1997) J. Mol. Biol. 273:614-622; Cohen & Prusiner, Chapter 5: Structural Studies of Prion Proteins in PRION BIOLOGY AND DISEASES, ed. S. Prusiner, Cold Spring Harbor Laboratory Press, 1999, pp: 191-228). The structural changes appear to be followed by alterations in the biochemical properties: PrP^{C} is soluble in non-denaturing detergents, PrP^{Sc} is insoluble; PrP^{C} is readily digested by proteases, while PrP^{Sc} is partially resistant, resulting in the formation of an N-terminally truncated fragment known as “PrPres” (Baldwin et al. (1995); Cohen & Prusiner (1995); Safar et al. (1998) Nat. Med. 4(10):1157-1165), “PrP 27-30” (27-30 kDa) or “K-resistant” (proteinase K resistant) form. In addition, PrP^{Sc} can convert PrP^{C} to the pathogenic conformation. See, e.g., Kaneko et al. (1995) Proc. Nat'l Acad. Sci. USA 92:11160-11164; Caughey (2003) Br Med Bull. 66:109-20.

Detection of the pathogenic isoforms of conformational disease proteins in living subjects and samples obtained from living subjects has proven difficult. Thus, definitive diagnosis and palliative treatments for these transmissible and amyloid containing conditions before death of the subject remains a substantially unmet challenge. Histopathological examination of brain biopsies is risky to the subject and lesions and amyloid deposits can be missed depending on where the biopsy sample is taken from. However, there are still risks involved with biopsies to animals, patients, and health care personnel. Further, the results from brain tests on animals are not usually obtained until the animal has entered the food supply. In addition, antibodies generated against prion peptides generally recognize both denatured PrP^{Sc} and PrP^{C} but are unable to selectively recognize infectious (undenatured) PrP^{Sc}. (See, e.g., Matsunaga et al. (2001) PROTEINS: Structure, Function and Genetics 44:110-118). Recently, antibodies that recognize the native Prp^{sc} form have been reported (see, U.S. Pat. Nos. 5,846,533 and 6,765,088)

Isolation of the PrP^{sc} in its native form for the generation of Prp^{sc}-specific antibodies, as a therapeutic target, for structural/mechanistic studies and as a positive control for prion detection assays (see U.S. Pat. No. 6,962,975) would be useful. The present invention describes a simple method for the isolation of this prion isoform.

### SUMMARY OF THE INVENTION

The present invention relates, in part, to peptide reagents that interact with prion proteins. More specifically, the peptide reagents (as described in International Application number PCT/US2004/026363, filed Aug. 13, 2004 and as further described herein) interact preferentially with the pathogenic isoforms of prion proteins. These peptide reagents can be used in a wide range of applications, including as tools to isolate pathogenic prions. For example, peptide reagents that interact preferentially with PrP^{Sc} as compared to PrP^{C} are useful for isolation of PrP^{Sc} in its native form. Thus the present invention provides a method for isolation of pathogenic prion proteins in the native conformation. Because the peptide reagents interact preferentially with the pathogenic prion proteins, the peptide reagent-pathogenic prion complexes can readily be separated from the non-pathogenic prion protein or denatured prion protein that may be present in a sample. The present inventors have developed methods described herein for dissociating the pathogenic prion from the complex with the peptide reagent under non-denaturing conditions. The pathogenic prion protein can thus readily be prepared in the native (i.e., infectious) conformation. It should be noted that the term “pathogenic prion” implies that the prion protein is in the native infectious conformation and the use of the redundant phrase “pathogenic prion proteins in the native conformation” is merely for emphasis and is not intended to suggest that the pathogenic prion can be in other than the native infectious conformation. Isolation of non-denatured PrP^{Sc} allows for, among other things, production of antibodies directed against PrP^{Sc} and screening of potential therapeutics. Isolation of native form PrP^{Sc} also facilitates structural studies of these proteins. Prior to the present disclosure, it has proven difficult to isolate significant quantities of native PrP^{Sc}.

The present invention provides a method for isolating a pathogenic prion protein in the native conformation by contacting a sample containing the pathogenic prion protein with a peptide reagent described herein that interacts preferentially with the pathogenic prion protein to form a complex and removing any unbound sample materials. The pathogenic prion can be further isolated by dissociating the complex under non-denaturing conditions. In preferred embodiments, the peptide reagent is provided on a solid support.

Thus, the present invention provides methods for isolating a pathogenic prion in its native form comprising: providing a solid support comprising one or more peptide reagents of the invention, contacting the solid support with a sample known or suspected of containing a pathogenic prion under conditions that allow the binding of the pathogenic prion, if present, to the peptide reagent; and removing any unbound sample materials. Additional embodiments further comprise the step of dissociating the bound pathogenic prion from the peptide reagent, and optionally, recovering the dissociated pathogenic prion.

The peptide reagents include those described in International Application number PCT/US2004/026363, filed Aug. 13, 2004, and U.S. application Ser. No. 11/056,950, filed Feb. 11, 2005, both of which applications are incorporated herein by reference. As described therein, the peptide reagents may be partially or fully synthetic, for example, may comprise one or more the following moieties: cyclized residues or peptides, multimers of peptides, labels, and/or other chemical moieties.

Examples of suitable peptide reagents include those derived from peptides of SEQ ID NOs:12 to 260, for example, peptides such as those depicted in SEQ ID NOs: 133 to 260, inclusive, and analogs and derivatives thereof. The peptide reagents described herein may interact with any conformational disease proteins, for example, prion proteins (e.g., the pathogenic protein PrP^{Sc}, and the nonpathogenic form PrP^{C}). In certain embodiments, peptide reagents interact preferentially with PrP^{Sc} as compared to PrP^{C}. The peptide reagents will generally be specific for PrP^{Sc} from more than one species, but may be specific for PrP^{Sc} from a single species.

In another embodiment, peptide reagents derived from peptides shown in any of sequences described herein are provided. In certain embodiments, the peptide reagents are derived from regions of a prion protein, for example, those regions corresponding to residues 23-43 or 85-156 (e.g., 23-30, 86-111, 89-112, 97-107, 113-135, and 136-156 numbered according to the mouse prion sequence shown in SEQ ID NO:2) are employed. For convenience, the amino acid residue numbers set out above are those corresponding to the mouse prion protein sequence in SEQ ID NO:2; one of ordinary skill in the art could readily identify corresponding regions in prion proteins of other species based on the sequences known in the art and the teachings provided herein. Exemplary peptide reagents include those derived from peptides having SEQ ID NO: 66, 67, 68, 72, 81, 96, 97, 98, 107, 108, 119, 120, 121, 122, 123, 124, 125, 126, 127, 133, 134, 135 133, 134, 135, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 249, 250, 251, 252, 253, 254, 255, or 256; or from peptides having SEQ ID NO: 14, 35, 36, 37, 40, 50, 51, 77, 89, 100, 101, 109, 110, 111, 112, 113, 114, 115, 116, 117, 118, 129, 130, 131, 132, 128, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 247, 257, 258, 259, or 260; or from peptides having SEQ ID NO: 56, 57, 65, 82, 84, or 136.

Any of the peptide reagents and/or antibodies described herein may be encoded for, in whole or in part, by one or more polynucleotides, which also form part of the present invention.

In one embodiment, the method comprises contacting a sample suspected of containing a pathogenic prion with one or more of the peptide reagents described herein under conditions that allow the interaction of the peptide reagent(s) and the pathogenic prion, if present, and subsequently separating the pathogenic prion from the peptide reagent under conditions whereby the pathogenic prion remains in its native form. The interaction of the peptide reagent(s) and the pathogenic prion can be carried out in solution, or one or more of the reactants can be provided in or on a solid phase. Similarly, the separation (elution) step may can be carried out in solution, or in or on a solid phase.

In one aspect of this embodiment, one or more peptide reagents of the present invention is provided on a solid support and contacted with a sample suspected of containing a pathogenic prion, under conditions that allow binding of the pathogenic prion, if present, to the peptide reagent. Unbound sample materials, including any non-pathogenic prion, can be removed and the pathogenic prion can be isolated, either while remaining bound to the peptide reagent or after dissociation from the peptide reagent.

In all of the foregoing embodiments providing a solid support comprising one or more peptide reagents of the invention, alternative embodiments are contemplated in which the peptide reagent is contacted with the sample prior to the peptide reagent being attached to the solid support. In these embodiments, the peptide reagent comprises one member of a binding pair and the solid support comprises the second member of the binding pair. For example, the peptide reagent of the invention may contain or be modified to contain biotin. The biotinylated peptide reagent is contacted with a sample suspected to contain a pathogenic prion under conditions to allow binding of the peptide reagent to the pathogenic prion. A solid support comprising avidin or streptavidin is then contacted with the biotinylated peptide reagent. Other suitable binding pairs are described herein.

In any of the methods using a solid support described herein, the solid support can be, for example, nitrocellulose, polystyrene, polypropylene, latex, polyvinyl fluoride, diazotized paper, nylon membranes, activated beads, and/or magnetically responsive beads, polyvinylchloride; polypropylene, polystyrene latex, polycarbonate, nylon, dextran, chitin, sand, silica, pumice, agarose, cellulose, glass, metal, polyacrylamide, silicon, rubber, polysaccharides; diazotized paper; activated beads, magnetically responsive beads, and any materials commonly used for solid phase synthesis, affinity separations, purifications, hybridization reactions, immunoassays and other such applications. The support can be particulate or can be in the form of a continuous surface and includes membranes, mesh, plates, pellets, slides, disks, capillaries, hollow fibers, needles, pins, chips, solid fibers, gels (e.g. silica gels) and beads, (e.g., pore-glass beads, silica gels, polystyrene beads optionally cross-linked with divinylbenzene, grafted co-poly beads, polyacrylamide beads, latex beads, dimethylacrylamide beads optionally crosslinked with N—N′-bis-acryloylethylenediamine, iron oxide magnetic beads, and glass particles coated with a hydrophobic polymer).

In addition, in any of the methods described herein the sample can be a biological sample, that is, a sample obtained or derived from a living or once-living organism, for example, organs, whole blood, blood fractions, blood components, plasma, platelets, serum, cerebrospinal fluid (CSF), brain tissue, nervous system tissue, muscle tissue, bone marrow, urine, tears, non-nervous system tissue, organs, and/or biopsies or necropsies. In preferred embodiments, the biological sample comprises blood, blood fractions or blood components. The sample may be a non-biological sample.

In another aspect, the invention includes various kits for isolating a pathogenic prion from a sample, the kit comprising: one or more of the peptide reagents described herein; and/or any of the solid supports comprising one or more of the peptide reagents described herein, optionally, reagents for dissociating the pathogenic prion from the peptide reagent (e.g., solutions of NaCl, KCl, Guanidinium isothiocyanate, or guanidinium hydrochloride of appropriate concentrations for use in the methods of the invention) and other necessary reagents and, optionally, positive and negative controls. The peptide reagent(s) may be detectably labeled.

These and other embodiments of the subject invention will readily occur to those of skill in the art in light of the disclosure herein.

### BRIEF DESCRIPTION OF THE DRAWINGS

FIG. 1 depicts the amino acid sequence of human (SEQ ID NO:1) and mouse (SEQ ID NO:2) prion proteins.

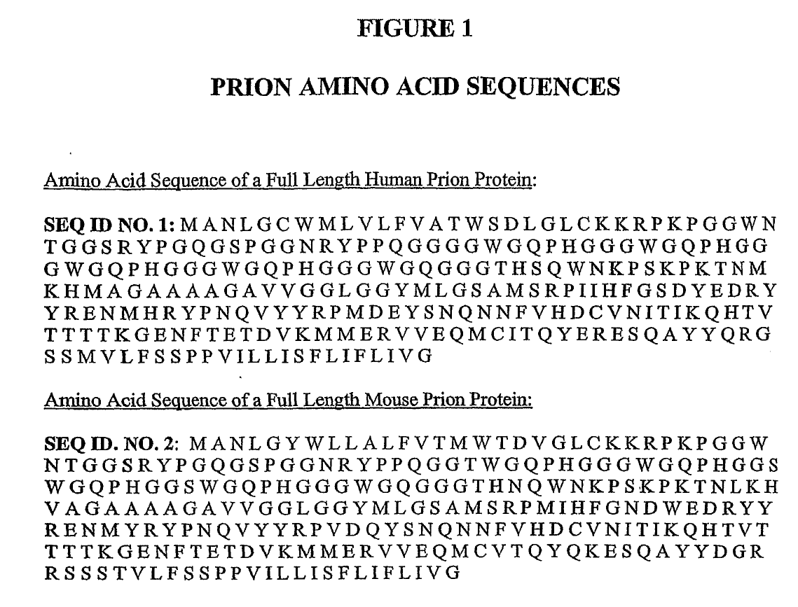

FIG. 2 depicts an alignment of prion proteins from human (SEQ ID NO:3), Syrian hamster (hamster) (SEQ ID NO:4), bovine (SEQ ID NO:5), sheep (SEQ ID NO:6), mouse (SEQ ID NO:7), elk (SEQ ID NO:8), fallow deer (fallow) (SEQ ID NO:9), mule deer (mule) (SEQ ID NO:10), and white tailed deer (white) (SEQ ID NO:11). Elk, Fallow Deer, Mule Deer, and White Tailed Deer only vary from each other at two residues, S/N128 and Q/E226 (shown in bold).

FIG. 3, panels A-F depict exemplary peptoid substitutions that may be made to prepare any of the peptide reagents described herein. The peptoids are circled in each panel and are shown in an exemplary peptide reagent as described herein (SEQ ID NO:14, QWNKPSKPKTNG), in which a proline residue (residue 8 of SEQ ID NO:14) is replaced with an N-substituted glycine (peptoid) residue. Panel A shows a peptide reagent in which a proline residue is substituted with the peptoid residue: N—(S)-(1-phenylethyl)glycine; panel B shows a peptide reagent in which a proline residue is substituted with the peptoid residue: N-(4-hydroxyphenyl)glycine; panel C shows a peptide reagent in which a proline residue is substituted with the peptoid residue: N-(cyclopropylmethyl)glycine; panel D shows a peptide reagent in which a proline residue is substituted with the peptoid residue: N-(isopropyl)glycine; panel E shows a peptide reagent in which a proline residue is substituted with the peptoid residue: N-(3,5-dimethoxybenzyl)glycine; and panel F shows a peptide reagent in which a proline residue is substituted with the peptoid residue: N-aminobutylglycine.

FIG. 4 depicts results of Western blotting experiments as described in Example 2. Lanes 1 and 2 show the presence of prion proteins in normal mouse brain homogenates (Lane 1, labeled “C”) and in denatured infected mouse brain homogenates (lane 2, labeled “Sc”). Lanes 3, 4 and 5 show specific binding of a peptide reagent as described herein (SEQ ID NO:68) to pathogenic prion forms in the presence of human plasma. In particular, Lane 3 is a human plasma control and lane 4 is a normal mouse brain homogenate sample. Lane 5 shows strong binding by the peptide reagent to PrP^{Sc} in infected mouse brain homogenate samples.

FIG. 5 depicts the structures of exemplary PEG-linked peptide reagents as described herein.

FIG. 6 depicts Western blot detection of PrP^{Sc} on peptide reagent-coated beads after treatment with chaotropic salts (guanidinium isothiocyanate (GdnSCN) or urea).

FIG. 7 depicts ELISA detection of denatured and non-denatured PrP^{Sc} dissociated from peptide reagent-coated beads treated with varying concentrations of chaotropic salts.

FIG. 8, panels A to D, are graphs depicting ELISA detection of PrP^{Sc} pulled down with peptide reagents as described herein and treated with increasing concentrations of chaotropic salts. In all panels, the black squares indicate treatment with chaotropic salts after pull-down with the peptide, followed by coating on non-treated ELISA plates. The open squares show ELISA detection when the plates were treated with 3M GdnSCN. FIG. 8A shows detection of PrP^{Sc} pulled down with GGGQWNKPSK*KTN, wherein * is N-(cyclopropylmethyl)glycine (SEQ ID NO:111) and treated with various concentrations of GdnSCN. FIG. 8B shows detection of PrP^{Sc} pulled down with GGGQWNKPSK*KTN, wherein * is N-(cyclopropylmethyl)glycine (SEQ ID NO:11) and treated with various concentrations of GdnHCl. FIG. 8C shows detection of PrP^{Sc} pulled down with GGGKKRPKPGG (SEQ ID NO:68) and treated with various concentrations of GdnSCN. FIG. 8D shows detection of PrP^{Sc} pulled down with GGGKKRPKPGG (SEQ ID NO:68) and treated with various concentrations of GdnHCl.

### DETAILED DESCRIPTION

The invention relates to a method for isolating pathogenic prion proteins in the native (i.e., infectious) conformation. The invention relies in part on the surprising and unexpected discovery that relatively small peptides (less than 50 to 100 amino acids in length, preferably less than 50 amino acids in length and even more preferably less than about 30 amino acids in length) can be used to discriminate between nonpathogenic and pathogenic prion proteins. Thus, the present disclosure relates to the surprising finding that these peptides and derivatives thereof (collectively “peptide reagents”), bind pathogenic and nonpathogenic protein forms with different specificity and/or affinity and, accordingly, can be used, in and of themselves, as diagnostic/detection reagents or as components of therapeutic compositions. Prior to the present disclosure, it was believed that only larger molecules (e.g., antibodies, PrP^{C}, α-form rPrP and plasminogen) could be used to differentiate pathogenic and nonpathogenic forms. As such, previously described antigenic peptides were used to generate antibodies that were evaluated for their ability to discriminate between pathogenic and nonpathogenic forms. However, due to the relatively nonimmunogenic nature of prion proteins, it has proven difficult to generate antibodies specific for pathogenic forms. See, e.g., R. A. Williamson et al. “Antibodies as Tools to Probe Prion Protein Biology” in PRION BIOLOGY AND DISEASES, ed. S. Prusiner, Cold Spring Harbor Laboratory Press, 1999, pp: 717-741.

The discovery that certain peptides as described herein interact preferentially with pathogenic (PrP^{Sc}) prion proteins allows for the development of novel reagents for diagnostics, detection assays and therapeutics, inter alia. Thus, the invention relates to purification or isolation methods utilizing these peptide reagents. The ability to isolate PrP^{Sc} in native conformation facilitates a range of other uses including, but not limited to, antibody generation, screening for therapeutics, structural studies and the like. The present invention thus provides a method for isolating a pathogenic prion protein in the native conformation comprising the steps of: (a) contacting a sample containing a pathogenic prion protein with a peptide reagent as described herein under conditions that allow the binding of the pathogenic prion protein, if present in said sample, to the peptide reagent to form a first complex; and (b) removing unbound sample materials.

The invention also provides a method for isolating a pathogenic prion protein in the native conformation comprising the steps of: (a) contacting a sample containing a pathogenic prion protein with a peptide reagent as described herein under conditions that allow the binding of the pathogenic prion protein, if present in said sample, to the peptide reagent to form a first complex; (b) removing unbound sample materials; and (c) dissociating the pathogenic prion protein from the peptide reagent under non-denaturing conditions.

The invention also provides a method of isolating a pathogenic prion protein in the native conformation, the method comprising the steps of: (a) providing a solid support comprising a first peptide reagent; (b) contacting the solid support with a sample under conditions which allow pathogenic prions, when present in the sample, to bind to the first peptide reagent; (c) removing unbound sample materials; and (d) dissociating the pathogenic prions from the solid support under non-denaturing conditions.

Any of the methods of isolating native conformation pathogenic prions may further comprise the step of separating the dissociated pathogenic prions from the peptide reagent, for example by centrifugation, precipitation, removal of the peptide by magnetic pull down, etc.

Furthermore, in any of the methods of isolating pathogenic prion proteins in their native conformation described herein, the dissociation step can comprise contacting the pathogenic prion-peptide reagent complex with a non-denaturing concentration of a salt or a chaotropic agent. The salt may be, for example, NaCl or KCl. The chaotropic agent may be, for example, GdnSCN or GdnHCl. In preferred embodiments, the concentration of salt is between about 0.5 M and 2 M, more preferably between about 1.0 M and about 1.5 M, and even more preferably, 1.0 M, 1.1 M, 1.2 M, 1.3 M, or 1.4 M. In preferred embodiments using GdnSCN or GdnHCl, the concentration of chaotropic agent is between about 0.4 M and 2 M, more preferably between about 0.4 M and about 1.0 M, and even more preferably, 0.5 M, 0.6 M, 0.7 M, 0.8 M, 0.9 M or 1.0 M. Alternatively or in addition to use of salts and chaotropic agents, centrifugation and/or precipitation steps may also be used to isolate non-denatured pathogenic prion proteins.

The peptide reagents used in the invention comprise a peptide that interacts preferentially with pathogenic isoforms as compared to nonpathogenic isoforms. For example, in certain embodiments, peptide reagents as described herein specifically bind to pathogenic conformational disease protein forms and do not bind (or bind to a lesser extent) to non-pathogenic forms.

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Handbook of Surface and Colloidal Chemistry (Birdi, K. S. ed., CRC Press, 1997); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Peters and Dalrymple, Fields Virology (2d ed), Fields et al. (eds.), B. N. Raven Press, New York, N.Y.

It is understood that the peptide reagents, antibodies and methods of this invention are not limited to particular formulations or process parameters as such may, of course, vary. It is also to be understood that the terminology used herein is for the purpose of describing particular embodiments of the invention only, and is not intended to be limiting.

All publications, patents and patent applications cited herein are hereby incorporated by reference in their entirety.

### I. DEFINITIONS

In order to facilitate an understanding of the invention, selected terms used in the application will be discussed below.

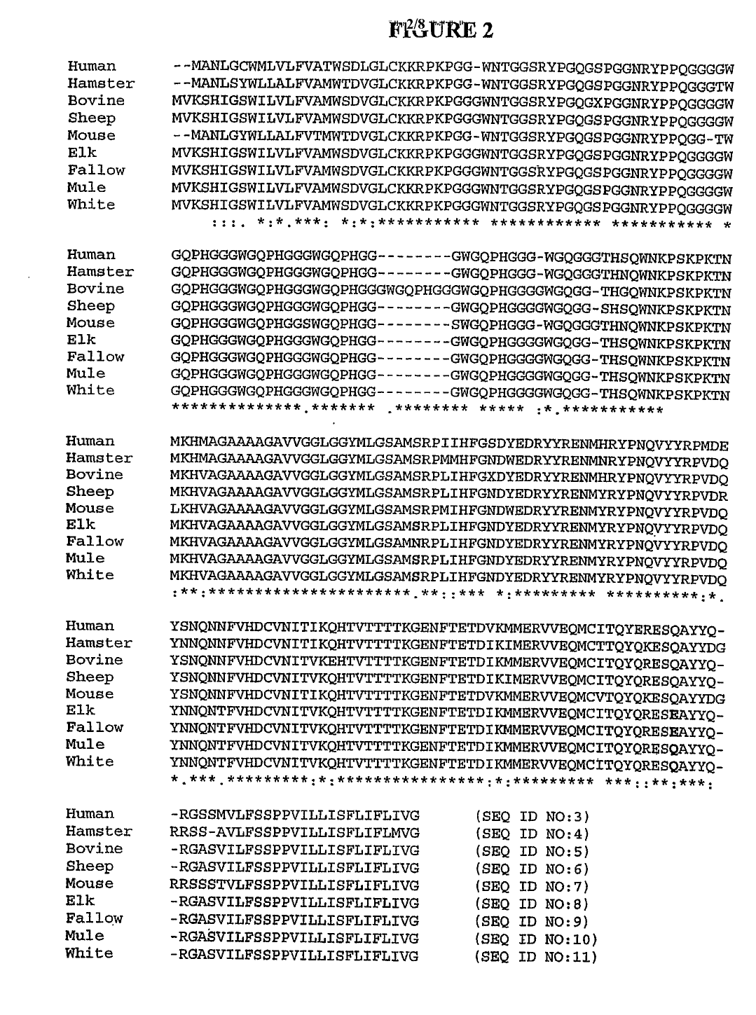

The terms “prion”, “prion protein”, “PrP protein” and “PrP” are used interchangeably herein to refer to both the pathogenic protein form (variously referred to as scrapie protein, pathogenic protein form, pathogenic isoform, pathogenic prion and PrP^{Sc}) and the non-pathogenic form (variously referred to as cellular protein form, cellular isoform, nonpathogenic isoform, nonpathogenic prion protein, and PrP^{C}), as well as the denatured form and various recombinant forms of the prion protein which may not have either the pathogenic conformation or the normal cellular conformation. The native pathogenic protein form (PrP^{Sc}) is associated with disease state (spongiform encephalopathies) in humans and animals; the non-pathogenic form is normally present in animal cells and may, under appropriate conditions, be converted to the pathogenic PrP^{Sc} conformation. Prions are naturally produced in a wide variety of mammalian species, including human, sheep, cattle, and mice. A representative amino acid sequence of a human prion protein is set forth as SEQ ID NO:1. A representative amino acid sequence of a mouse prion protein is set forth as SEQ ID NO:2. Other representative sequences are shown in FIG. 2.

As used herein, the term “pathogenic” may mean that the protein actually causes the disease or it may simply mean that the protein is associated with the disease and therefore is present when the disease is present. Thus, a pathogenic protein as used in connection with this disclosure is not necessarily a protein that is the specific causative agent of a disease. Pathogenic forms may or may not be infectious. The term “pathogenic prion form” is used more specifically to refer to the conformation and/or the beta-sheet-rich conformation of mammalian, avian or recombinant prion proteins. Generally, the beta-sheet-rich conformation is proteinase K resistant. The terms “non-pathogenic” and “cellular” when used with respect to conformational disease protein forms are used interchangeably to refer to the normal isoform of the protein whose presence is not associated with sickness.

Furthermore, a “prion protein” or “conformational disease protein” as used herein is not limited to a polypeptide having the exact sequence to those described herein. It is readily apparent that the terms encompass conformational disease proteins from any of the identified or unidentified species or diseases (e.g., Alzheimer's, Parkinson's, etc.). One of ordinary skill in the art in view of the teachings of the present disclosure and the art can determine regions corresponding to the sequences shown in the Figures in any other prion proteins, using for example, sequence comparison programs (e.g., BLAST and others described herein) or identification and alignment of structural features or motifs.

The term “PrP gene” is used herein to describe any genetic material that expresses prion proteins including known polymorphisms and pathogenic mutations. The term “PrP gene” refers generally to any gene of any species that encodes any form of a PrP protein. Some commonly known PrP sequences are described in Gabriel et al., Proc. Natl. Acad. Sci. USA 89:9097-9101 (1992), and U.S. Pat. Nos. 5,565,186; 5,763,740; 5,792,901; and WO97/04814, incorporated herein by reference to disclose and describe such sequences. The PrP gene can be from any animal, including the “host” and “test” animals described herein and any and all polymorphisms and mutations thereof, it being recognized that the terms include other such PrP genes that are yet to be discovered. The protein expressed by such a gene can assume either a PrP^{C} (non-disease) or PrP^{Sc} (disease) form.

“Prion-related disease” as used herein refers to a disease caused in whole or in part by a pathogenic prion protein (PrP^{Sc}). Prion-related diseases include, but are not limited to, scrapie, bovine spongiform encephalopathies (BSE), mad cow disease, feline spongiform encephalopathies, kuru, Creutzfeldt-Jakob Disease (CJD), new variant Creutzfeldt-Jakob Disease (nvCJD), chronic wasting disease (CWD), Gerstmann-Strassler-Scheinker Disease (GSS), and fatal familial insomnia (FFI).

The term “peptide reagent” as used herein generally refers to any compound comprising naturally occurring or synthetic polymers of amino acid or amino acid-like molecules, including but not limited to compounds comprising only amino and/or imino molecules. The peptide reagents of the present invention interact preferentially with a pathogenic prion protein and are typically derived from fragments of a prion protein. The term “peptide” will be used interchangeably with “oligopeptide” or “polypeptide” and no particular size is implied by use of these terms Included within the definition are, for example, peptides containing one or more analogs of an amino acid (including, for example, unnatural amino acids, peptoids, etc.), peptides with substituted linkages, as well as other modifications known in the art, both naturally occurring and non-naturally occurring (e.g., synthetic). Thus, synthetic peptides, dimers, multimers (e.g., tandem repeats, multiple antigenic peptide (MAP) forms, linearly-linked peptides), cyclized, branched molecules and the like, are included within the definition. The terms also include molecules comprising one or more N-substituted glycine residues (a “peptoid”) and other synthetic amino acids or peptides. (See, e.g., U.S. Pat. Nos. 5,831,005; 5,877,278; and 5,977,301; Nguyen et al. (2000) Chem Biol. 7(7):463-473; and Simon et al. (1992) Proc. Natl. Acad. Sci. USA 89(20):9367-9371 for descriptions of peptoids). Non-limiting lengths of peptides suitable for use in the present invention includes peptides of 3 to 5 residues in length, 6 to 10 residues in length (or any integer therebetween), 11 to 20 residues in length (or any integer therebetween), 21 to 75 residues in length (or any integer therebetween), 75 to 100 (or any integer therebetween), or polypeptides of greater than 100 residues in length. Typically, peptides useful in this invention can have a maximum length suitable for the intended application. Preferably, the peptide is between about 3 and 100 residues in length. Generally, one skilled in art can easily select the maximum length in view of the teachings herein. Further, peptide reagents as described herein, for example synthetic peptides, may include additional molecules such as labels, linkers, or other chemical moieties (e.g., biotin, amyloid specific dyes such as Control Red or Thioflavin). Such moieties may further enhance interaction of the peptides with the prion proteins and/or further detection of prion proteins.

Peptide reagents also includes derivatives of the amino acid sequences of the invention having one or more substitution, addition and/or deletion, including one or more non-naturally occurring amino acid. Preferably, derivatives exhibit at least about 50% identity to any wild type or reference sequence, preferably at least about 70% identity, more preferably at least about 75%, 80%, 85%, 90%, 91%, 92%, 93%, 94%, 95%, 96%, 97%, 98%, 99% or 100% sequence identity to any wild type or reference sequence described herein. Sequence (or percent) identity can be determined as described below. Such derivatives can include postexpression modifications of the polypeptide, for example, glycosylation, acetylation, phosphorylation, and the like.

Peptide derivatives can also include modifications to the native sequence, such as deletions, additions and substitutions (generally conservative in nature), so long as the polypeptide maintains the desired activity. These modifications may be deliberate, as through site-directed mutagenesis, or may be accidental, such as through mutations of hosts that produce the proteins or errors due to PCR amplification. Furthermore, modifications may be made that have one or more of the following effects: reducing toxicity; increasing affinity and/or specificity for prion proteins; facilitating cell processing (e.g., secretion, antigen presentation, etc.); and facilitating presentation to B-cells and/or T-cells. Polypeptides described herein can be made recombinantly, synthetically, purified from natural sources, or in tissue culture.

A “fragment” as used herein refers to a peptide consisting of only a part of the intact full-length protein and structure as found in nature. For instance, a fragment can include a C-terminal deletion and/or an N-terminal deletion of a protein. Typically, the fragment retains one, some or all of the functions of the full-length polypeptide sequence from which it is derived. Typically, a fragment will comprise at least 5 consecutive amino acid residues of the native protein; preferably, at least about 8 consecutive amino acid residues; more preferably, at least about 10, 11, 12, 13, 14, 15, 16, 17, 18, 19, 20, 21, 22, 23, 24, 25, 26, 27, 28, 29, or 30 consecutive amino acid residues of the native protein.

The term “polynucleotide”, as known in the art, generally refers to a nucleic acid molecule. A “polynucleotide” can include both double- and single-stranded sequences and refers to, but is not limited to, prokaryotic sequences, eukaryotic mRNA, cDNA from viral, prokaryotic or eukaryotic mRNA, genomic RNA and DNA sequences from viral (e.g. RNA and DNA viruses and retroviruses), prokaryotic DNA or eukaryotic (e.g., mammalian) DNA, and especially synthetic DNA sequences. The term also captures sequences that include any of the known base analogs of DNA and RNA, and includes modifications such as deletions, additions and substitutions (generally conservative in nature), to the native sequence. These modifications may be deliberate, as through site-directed mutagenesis, or may be accidental, such as through mutations of hosts including prion-encoding polynucleotides. Modifications of polynucleotides may have any number of effects including, for example, facilitating expression of the polypeptide product in a host cell.

A polynucleotide can encode a biologically active (e.g., immunogenic or therapeutic) protein or polypeptide. Depending on the nature of the polypeptide encoded by the polynucleotide, a polynucleotide can include as little as 10 nucleotides, e.g., where the polynucleotide encodes an antigen or epitope. Typically, the polynucleotide encodes peptides of at least 18, 19, 20, 21, 22, 23, 24, 25, 30 or even more amino acids.

A “polynucleotide coding sequence” or a sequence that “encodes” a selected polypeptide, is a nucleic acid molecule that is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A transcription termination sequence may be located 3′ to the coding sequence. Typical “control elements,” include, but are not limited to, transcription regulators, such as promoters, transcription enhancer elements, transcription termination signals, and polyadenylation sequences; and translation regulators, such as sequences for optimization of initiation of translation, e.g., Shine-Dalgarno (ribosome binding site) sequences, Kozak sequences (i.e., sequences for the optimization of translation, located, for example, 5′ to the coding sequence), leader sequences (heterologous or native), translation initiation codon (e.g., ATG), and translation termination sequences. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is induced by an analyte, cofactor, regulatory protein, etc.), and constitutive promoters.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

A “recombinant” nucleic acid molecule as used herein to describe a nucleic acid molecule means a polynucleotide|of genomic, cDNA, semi synthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

By “isolated” is meant, when referring to a polynucleotide or a polypeptide, that the indicated molecule is separate and discrete from the whole organism with which the molecule is found in nature or, when the polynucleotide or polypeptide is not found in nature, is sufficiently free of other biological macromolecules so that the polynucleotide or polypeptide can be used for its intended purpose.

“Antibody” as known in the art includes one or more biological moieties that, through chemical or physical means, can bind to or associate with an epitope of a polypeptide of interest. For example, the antibodies of the invention may interact preferentially with (e.g., specifically bind to) pathogenic prion conformations. The term “antibody” includes antibodies obtained from both polyclonal and monoclonal preparations, as well as the following: hybrid (chimeric) antibody molecules (see, for example, Winter et al. (1991) Nature 349: 293-299; and U.S. Pat. No. 4,816,567; F(ab′)₂ and F(ab) fragments; F^{v} molecules (non-covalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5897-5883); dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); and, any functional-fragments obtained from such molecules, wherein such fragments retain immunological binding properties of the parent antibody molecule. The term “antibody” further includes antibodies obtained through non-conventional processes, such as phage display.

As used herein, the term “monoclonal antibody” refers to an antibody composition having a homogeneous antibody population. The term is not limited regarding the species or source of the antibody, nor is it intended to be limited by the manner in which it is made. Thus, the term encompasses antibodies obtained from murine hybridomas, as well as human monoclonal antibodies obtained using human rather than murine hybridomas. See, e.g., Cote, et al. Monoclonal Antibodies and Cancer Therapy, Alan R. Liss, 1985, p 77.

If polyclonal antibodies are desired, a selected mammal (e.g., mouse, rabbit, goat, horse, etc.) is generally immunized with an immunogenic composition (e.g., a peptide reagent as described herein). Serum from the immunized animal is collected and treated according to known procedures. If serum containing polyclonal antibodies to the selected peptide reagent contains antibodies to other antigens, the polyclonal antibodies can be purified by immunoaffinity chromatography. Techniques for producing and processing polyclonal antisera are known in the art, see for example, Mayer and Walker, eds. (1987) IMMUNOCHEMICAL METHODS IN CELL AND MOLECULAR BIOLOGY (Academic Press, London).

One skilled in the art can also readily produce monoclonal antibodies directed against peptide reagents described herein. The general methodology for making monoclonal antibodies by hybridomas is well known. Immortal antibody-producing cell lines can be created by cell fusion, and also by other techniques such as direct transformation of B-lymphocytes with oncogenic DNA, or transfection with Epstein-Barr virus. See, e.g., M. Schreier et al. (1980) HYBRIDOMA TECHNIQUES; Hammerling et al. (1981), MONOCLONAL ANTIBODIES AND T-CELL HYBRIDOMAS; Kennett et al. (1980) MONOCLONAL ANTIBODIES; see also, U.S. Pat. Nos. 4,341,761; 4,399,121; 4,427,783; 4,444,887; 4,466,917; 4,472,500; 4,491,632; and 4,493,890.

As used herein, a “single domain antibody” (dAb) is an antibody that is comprised of an VH domain, which binds specifically with a designated antigen. A dAb does not contain a VL domain, but may contain other antigen binding domains known to exist to antibodies, for example, the kappa and lambda domains. Methods for preparing dabs are known in the art. See, for example, Ward et al, Nature 341: 544 (1989).

Antibodies can also be comprised of VH and VL domains, as well as other known antigen binding domains. Examples of these types of antibodies and methods for their preparation are known in the art (see, e.g., U.S. Pat. No. 4,816,467, which is incorporated herein by reference), and include the following. For example, “vertebrate antibodies” refers to antibodies that are tetramers or aggregates thereof, comprising light and heavy chains which are usually aggregated in a “Y” configuration and which may or may not have covalent linkages between the chains. In vertebrate antibodies, the amino acid sequences of the chains are homologous with those sequences found in antibodies produced in vertebrates, whether in situ or in vitro (for example, in hybridomas). Vertebrate antibodies include, for example, purified polyclonal antibodies and monoclonal antibodies, methods for the preparation of which are described infra.

“Hybrid antibodies” are antibodies where chains are separately homologous with reference to mammalian antibody chains and represent novel assemblies of them, so that two different antigens are precipitable by the tetramer or aggregate. In hybrid antibodies, one pair of heavy and light chains are homologous to those found in an antibody raised against a first antigen, while a second pair of chains are homologous to those found in an antibody raised against a second antibody. This results in the property of “divalence”, i.e., the ability to bind two antigens simultaneously. Such hybrids can also be formed using chimeric chains, as set forth below.

“Chimeric antibodies” refers to antibodies in which the heavy and/or light chains are fusion proteins. Typically, one portion of the amino acid sequences of the chain is homologous to corresponding sequences in an antibody derived from a particular species or a particular class, while the remaining segment of the chain is homologous to the sequences derived from another species and/or class. Usually, the variable region of both light and heavy chains mimics the variable regions or antibodies derived from one species of vertebrates, while the constant portions are homologous to the sequences in the antibodies derived from another species of vertebrates. However, the definition is not limited to this particular example. Also included is any antibody in which either or both of the heavy or light chains are composed of combinations of sequences mimicking the sequences in antibodies of different sources, whether these sources be from differing classes or different species of origin, and whether or not the fusion point is at the variable/constant boundary. Thus, it is possible to produce antibodies in which neither the constant nor the variable region mimic known antibody sequences. It then becomes possible, for example, to construct antibodies whose variable region has a higher specific affinity for a particular antigen, or whose constant region can elicit enhanced complement fixation, or to make other improvements in properties possessed by a particular constant region.

Another example is “altered antibodies”, which refers to antibodies in which the naturally occurring amino acid sequence in a vertebrate antibody has been varies. Utilizing recombinant DNA techniques, antibodies can be redesigned to obtain desired characteristics. The possible variations are many, and range from the changing of one or more amino acids to the complete redesign of a region, for example, the constant region. Changes in the constant region, in general, to attain desired cellular process characteristics, e.g., changes in complement fixation, interaction with membranes, and other effector functions. Changes in the variable region can be made to alter antigen-binding characteristics. The antibody can also be engineered to aid the specific delivery of a molecule or substance to a specific cell or tissue site. The desired alterations can be made by known techniques in molecular biology, e.g., recombinant techniques, site-directed mutagenesis, etc.

Yet another example are “univalent antibodies”, which are aggregates comprised of a heavy-chain/light-chain dimer bound to the Fc (i.e., stem) region of a second heavy chain. This type of antibody escapes antigenic modulation. See, e.g., Glennie et al. Nature 295: 712 (1982). Included also within the definition of antibodies are “Fab” fragments of antibodies. The “Fab” region refers to those portions of the heavy and light chains which are roughly equivalent, or analogous, to the sequences which comprise the branch portion of the heavy and light chains, and which have been shown to exhibit immunological binding to a specified antigen, but which lack the effector Fc portion. “Fab” includes aggregates of one heavy and one light chain (commonly known as Fab′), as well as tetramers containing the 2H and 2 L chains (referred to as F(ab)2), which are capable of selectively reacting with a designated antigen or antigen family. Fab antibodies can be divided into subsets analogous to those described above, i.e., “vertebrate Fab”, “hybrid Fab”, “chimeric Fab”, and “altered Fab”. Methods of producing Fab fragments of antibodies are known within the art and include, for example, proteolysis, and synthesis by recombinant techniques.

“Antigen-antibody complex” refers to the complex formed by an antibody that is specifically bound to an epitope on an antigen.

A peptide (or peptide reagent) is said to “interact” with another peptide or protein if it binds specifically, non-specifically or in some combination of specific and non-specific binding. A peptide (or peptide reagent) is said to “interact preferentially” with a pathogenic prion protein if it bind with greater affinity and/or greater specificity to the pathogenic form than to nonpathogenic isoforms. A peptide reagent that interacts preferentially with a pathogenic prion protein is also referred to herein as a pathogenic prion-specific peptide reagent. It is to be understood that a preferential interaction does not necessarily require interaction between specific amino acid residues and/or motifs of each peptide. For example, in certain embodiments, the peptide reagents described herein interact preferentially with pathogenic isoforms but, nonetheless, may be capable of binding nonpathogenic isoforms at a weak, yet detectable, level (e.g., 10% or less of the binding shown to the polypeptide of interest). Typically, weak binding, or background binding, is readily discernible from the preferentially interaction with the compound or polypeptide of interest, e.g., by use of appropriate controls. In general, peptides of the invention bind pathogenic prions in the presence of 10⁶-fold excess of nonpathogenic forms.

The term “affinity” refers to the strength of binding and can be expressed quantitatively as a dissociation constant (K_{d}). Preferably, a peptide (or peptide reagent) that interacts preferentially with a pathogenic isoform preferably interacts with the pathogenic isoform with at least 2 fold greater affinity, more preferably at least 10 fold greater affinity and even more preferably at least 100 fold greater affinity than it interacts with the nonpathogenic isoform. Binding affinity (i.e., K_{d}) can be determined using standard techniques.

Techniques for determining amino acid sequence “similarity” or “percent identity” are well known in the art. In general, “similarity” means the amino acid to amino acid comparison of two or more polypeptides at the appropriate place, where amino acids are identical or possess similar chemical and/or physical properties such as charge or hydrophobicity. A so-termed “percent identity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby, and comparing this to a second amino acid sequence. In general, “identity” refers to an exact nucleotide to nucleotide or amino acid to amino acid correspondence of two polynucleotides or polypeptide sequences, respectively.

Two or more amino acid or polynucleotide sequences can be compared by determining their “percent identity.” Percent identity can be determined by a direct comparison of the sequence information between two molecules (the reference sequence and a sequence with unknown % identity to the reference sequence) by aligning the sequences, counting the exact number of matches between the two aligned sequences, dividing by the length of the reference sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH™ package of programs copyrighted by the University of Edinburgh, developed by John F. Collins and Shane S. Sturrok, and available from numerous sources, for example on the internet. From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

An “immunogenic composition” as used herein refers to any composition (e.g., peptide, antibody and/or polynucleotides) where administration of the composition to a subject results in the development in the subject of a humoral and/or a cellular immune response. The immunogenic composition can be introduced directly into a recipient subject, such as by injection, inhalation, oral, intranasal or any other parenteral or mucosal (e.g., intra-rectally or intra-vaginally) route of administration.

By “epitope” is meant a site on an antigen to which specific B cells and/or T cells respond, rendering the molecule including such an epitope capable of eliciting an immunological reaction or capable of reacting with antibodies present in a biological sample. The term is also used interchangeably with “antigenic determinant” or “antigenic determinant site.” An epitope can comprise 3 or more amino acids in a spatial conformation unique to the epitope. Generally, an epitope consists of at least 5 such amino acids and, more usually, consists of at least 8-10 such amino acids. Methods of determining spatial conformation of amino acids are known in the art and include, for example, x-ray crystallography and 2-dimensional nuclear magnetic resonance. Furthermore, the identification of epitopes in a given protein is readily accomplished using techniques well known in the art, such as by the use of hydrophobicity studies and by site-directed serology. See, also, Geysen et al., Proc. Natl. Acad. Sci. USA (1984) 81:3998-4002 (general method of rapidly synthesizing peptides to determine the location of immunogenic epitopes in a given antigen); U.S. Pat. No. 4,708,871 (procedures for identifying and chemically synthesizing epitopes of antigens); and Geysen et al., Molecular Immunology (1986) 23:709-715 (technique for identifying peptides with high affinity for a given antibody). Antibodies that recognize the same epitope can be identified in a simple immunoassay showing the ability of one antibody to block the binding of another antibody to a target antigen.

An “immunological response” or “immune response” as used herein is the development in the subject of a humoral and/or a cellular immune response to a peptide as described herein when the polypeptide is present in a vaccine composition. These antibodies may also neutralize infectivity, and/or mediate antibody-complement or antibody dependent cell cytotoxicity to provide protection to an immunized host. Immunological reactivity may be determined in standard immunoassays, such as a competition assays, well known in the art.

“Gene transfer” or “gene delivery” refers to methods or systems for reliably inserting DNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from alphaviruses, pox viruses and vaccinia viruses. When used for immunization, such gene delivery expression vectors may be referred to as vaccines or vaccine vectors.

The term “sample” includes biological and non-biological samples. Biological samples are those obtained or derived from a living or once-living organism. Non-biological samples are not derived from living or once-living organisms. Biological samples include, but are not limited to, samples derived from an animal (living or dead) such as organs (e.g., brain, liver, kidney, etc), whole blood, blood fractions, plasma, cerebrospinal fluid (CSF), urine, tears, tissue, organs, biopsies. Examples of non-biological samples include pharmaceuticals, foods, cosmetics and the like.

The terms “label” and “detectable label” refer to a molecule capable of detection, including, but not limited to, radioactive isotopes, fluorescers, luminescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to fluorescein, rhodamine, dansyl, umbelliferone, Texas red, luminol, acridinium esters, NADPH, beta-galactosidase, horseradish peroxidase, glucose oxidase, alkaline phosphatase and urease. The label can also be an epitope tag (e.g., a His-His tag), an antibody or a amplifiable or otherwise detectable oligonucleotide.

### II. GENERAL OVERVIEW

Described herein are compositions comprising a peptide reagent (and/or polynucleotides encoding these peptide reagents) in which the peptide reagent is capable of distinguishing between pathogenic and nonpathogenic isoforms of prion proteins, for example by preferentially interacting with one form and not the other. Use of these peptide reagents for isolating PrP^{Sc} in its native (pathogenic) conformation is also described. Antibodies generated using these peptide reagents as well as compositions comprising and methods of making and using these peptide reagents and/or antibodies are also provided (e.g., for isolation and/or detection of the pathogenic prion protein).

The invention relies in part on the discovery by the present inventors that relatively small fragments of a prion protein can interact preferentially with the pathogenic form of the prion. These fragments need not be part of a larger protein structure or other type of scaffold molecule in order to exhibit this preferential interaction with the pathogenic prion isoform. While not wanting to be held to any particular theory, it appears that the peptide fragments spontaneously take on a conformation that allows binding to the pathogenic prion isoform but not to the nonpathogenic prion isoform, perhaps by mimicking a conformation that is present in the nonpathogenic isoform. This general principle, that certain fragments of a conformational disease protein interact preferentially with the pathogenic form of that conformational disease protein, here demonstrated for prions, can readily be applied to other conformational disease proteins to produce peptide reagents that interact preferentially with the pathogenic forms. It will be apparent to one of ordinary skill in the art that, while the fragments provide a starting point (in terms of size or sequence characteristics, for example), that many modifications can be made on the fragments to produce peptide reagents with more desirable attributes (e.g., higher affinity, greater stability, greater solubility, less protease sensitivity, greater specificity, easier to synthesize, etc.).

In general, the peptide reagents described herein are able to interact preferentially with pathogenic forms of prion proteins. Accordingly, these peptide reagents can be used as described herein to isolate (and detect the presence of) pathogenic prion proteins and, hence, diagnosis of prion-related diseases in virtually any sample, biological or non-biological, including living or dead brain, spinal cord, or other nervous system tissue as well as blood. Furthermore, isolation of PrP^{Sc} in its native form not only allows for diagnosis, but also for further uses of the previously difficult to isolate PrP^{Sc} conformation including drug screening (e.g., for therapeutics), structural studies, antibody production and the like.

Thus the invention provides a method for isolating a pathogenic prion in the native conformation comprising the steps of: (a) contacting a sample containing a pathogenic prion protein with a peptide reagent as described herein under conditions that allow the binding of the pathogenic prion protein, if present in said sample, to the peptide reagent to form a first complex; and (b) removing unbound sample materials.

The invention also provides a method for isolating a pathogenic prion protein in the native conformation comprising the steps of: (a) contacting a sample containing a pathogenic prion protein with a peptide reagent as described herein under conditions that allow the binding of the pathogenic prion protein, if present in said sample, to the peptide reagent to form a first complex; (b) removing unbound sample materials; and (c) dissociating the pathogenic prion protein from the peptide reagent under non-denaturing conditions.

The invention also provides a method of isolating a pathogenic prion protein in the native conformation, the method comprising the steps of: (a) providing a solid support comprising a first peptide reagent; (b) contacting the solid support with a sample under conditions which allow pathogenic prions, when present in the sample, to bind to the first peptide reagent; (c) removing unbound sample materials; and (d) dissociating the pathogenic prions from the solid support under non-denaturing conditions.

Any of the methods of isolating native conformation pathogenic prions may further comprise the step of separating the dissociated pathogenic prions from the peptide reagent, for example by centrifugation, precipitation, removal of the peptide by magnetic pull down, etc.

Furthermore, in any of the methods of isolating pathogenic prion proteins in their native conformation described herein, the dissociation step can comprise contacting the pathogenic prion-peptide reagent complex with a non-denaturing concentration of a salt or a chaotropic agent. The salt may be, for example, NaCl or KCl. The chaotropic agent may be, for example, GdnSCN or GdnHCl. In preferred embodiments, the concentration of salt is between about 0.5 M and 2 M, more preferably between about 1.0 M and about 1.5 M, and even more preferably, 1.0 M, 1.1 M, 1.2 M, 1.3 M, or 1.4 M. In preferred embodiments using GdnSCN or GdnHCl, the concentration of chaotropic agent is between about 0.4 M and 2 M, more preferably between about 0.4 M and about 1.0 M, and even more preferably, 0.5 M, 0.6 M, 0.7 M, 0.8 M, 0.9 M or 1.0 M. Alternatively or in addition to use of salts and chaotropic agents, centrifugation and/or precipitation steps may also be used to isolate non-denatured pathogenic prion proteins.

### III. A. PEPTIDE REAGENTS

Peptide reagents that interact preferentially with pathogenic prion proteins have been described in US application No. PCT/US2004/026363, filed Aug. 13, 2004 and U.S. application Ser. No. 11/056,950, filed Feb. 11, 2005. These peptide reagents are useful in the method of the present invention.

It should also be noted that prion proteins (and other conformational disease proteins) have two different 3-dimensional conformations with the same amino acid sequence. One conformation is associated with disease characteristics and is generally insoluble whereas the other conformation is not associated with disease characteristics and is soluble. See, e.g., Wille, et al., “Structural Studies of the Scrapie Prion Protein by Electron Crystallography”, Proc. Natl. Acad. Sci. USA, 99 (6): 3563-3568 (2002).

Thus, in certain aspects, the peptide reagents described herein comprise an amino acid sequence derived from a naturally-occurring protein, for example a conformational disease protein (e.g., prion protein) or a protein that contains motifs or sequences that exhibit homology to prion proteins. In particular, the peptide reagents of the invention are typically derived from a naturally-occurring prion protein. The peptide reagents are preferably derived from the amino acid sequences from certain regions of the prion proteins. These preferred regions are exemplified with respect to the mouse prion sequence (SEQ ID NO:2), in regions from amino acid residue 23-43 and 85-156, and subregions thereof. The invention is not limited to peptide reagents derived from the mouse sequences but include peptide reagents derived in similar fashion as described herein, from prion sequences of any species, including human, bovine, sheep, deer, elk, hamster. When derived from prion proteins, the peptide reagents described herein may include a polyproline type II helix motif. This motif typically contains the general sequence PxxP (e.g., residues 102-105 of SEQ ID NO:1), although other sequences, in particular alanine tetrapeptides, have been suggested to form polyproline type II helices as well (see, e.g., Nguyen et al. Chem Biol. 2000 7:463; Nguyen et al. Science 1998 282:2088; Schweitzer-Stenner et al. J. Am. Chem Soc. 2004 126:2768). In the PxxP sequence, “x” can be any amino acid and “P” is proline in the naturally occurring sequence but may be replaced by a proline substitute in the peptide reagents of the invention. Such proline substitutes include N-substituted glycines commonly referred to as peptoids. Thus, in the peptide reagents of the invention that include a polyproline type II helix based on the PxxP sequence, “P” represents a proline or an N-substituted glycine residues and “x” represents any amino acid or amino acid analog. Particularly preferred N-substituted glycines are described herein.

Further, the polynucleotide and amino acid sequence for prion proteins produced by many different species are known, including human, mouse, sheep and cattle. Variants to these sequences also exist within each species. Thus, the peptide reagents used in the invention can comprise fragments or derivatives of the amino acid sequences of any species or variant. For example, in certain embodiments, the peptide reagents described herein are derived from any of the sequences set forth in FIG. 2 (SEQ ID NOs:3-11). The sequences of the peptide reagents that are specifically disclosed herein are generally based on the mouse prion sequence, however, one skilled in the art can readily substitute corresponding sequences from other species when appropriate. For example, if isolation of human pathogenic prion protein is desired, it may be preferable to utilize peptide reagents comprising or derived from the human prion sequences. Replacement of the mouse sequences with those of the corresponding human sequences can be easily done. In a particular example, in peptide reagents derived from the region from about residue 85 to about residue 112 (e.g., SEQ ID NO:35, 36, 37, 40), the leucine at position corresponding to residue 109 may be replaced with a methionine, the valine at position corresponding to residue 112 may be replaced with methionine, and the asparagine at position corresponding to 97 may be replaced with serine. Likewise, if a bovine pathogenic prion is desired, the appropriate substitutions may be made in the disclosed peptide sequences to reflect the bovine prion sequence. Thus, continuing with the above example for peptide reagents derived from the region from about residue 85 to about residue 112, the leucine at position corresponding to residue 109 may be replaced with a methionine and the asparagine at position corresponding to 97 may be replaced with glycine. Derivatives of prion proteins, including amino acid replacements, deletions, additions and other mutations to these sequences can also be used. Preferably, any amino acid replacements, additions, and deletions as compared to a prion protein sequence do not affect the ability of the peptide reagent to interact with pathogenic form. In many instances, however, it is not absolutely required that the peptide reagent have sequences derived from the same species as the prion protein to be isolated as most of the peptide reagents described will interact preferentially with pathogenic prions from many different species.

It should be understood that no matter what source is used for the peptide reagents described herein, these peptide reagents will not necessarily exhibit sequence identity to known prion proteins. Thus, the peptide reagents described herein can include one or more amino acid replacements, additions, and deletions relative to the naturally-occurring prion protein or the sequences disclosed herein, so long as they retain the ability to interact preferentially with pathogenic forms of conformational disease proteins. In certain embodiments, conservative amino acid replacements are preferred. Conservative amino acid replacements are those that take place within a family of amino acids that are related in their side chains. Genetically encoded amino acids are generally divided into four families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. For example, it is reasonably predictable that an isolated replacement of a leucine with an isoleucine or valine, an aspartate with a glutamate, a threonine with a serine, or a similar conservative replacement of an amino acid with a structurally related amino acid will not have a major effect on the biological activity.

It will also be apparent that any combination of the natural amino acids and non-natural amino acid analogs can be used to make the peptide reagents described herein. Commonly encountered amino acid analogs that are not gene-encoded include, but are not limited to, ornithine (Orn); aminoisobutyric acid (Aib); benzothiophenylalanine (BtPhe); albizziin (Abz); t-butylglycine (Tle); phenylglycine (PhG); cyclohexylalanine (Cha); norleucine (Nle); 2-naphthylalanine (2-Nal); 1-naphthylalanine (1-Nal); 2-thienylalanine (2-Thi); 1,2,3,4-tetrahydroisoquinoline-3-carboxylic acid (Tic); N-methylisoleucine (N-MeIle); homoarginine (Har); Na-methylarginine (N-MeArg); phosphotyrosine (pTyr or pY); pipecolinic acid (Pip); 4-chlorophenylalanine (4-ClPhe); 4-fluorophenylalanine (4-FPhe); 1-aminocyclopropanecarboxylic acid (1-NCPC); and sarcosine (Sar). Any of the amino acids used in the peptide reagents of the present invention may be either the D- or, more typically, L-isomer.

Other non-naturally occurring analogs of amino acids that may be used to form the peptide reagents described herein include peptoids and/or peptidomimetic compounds such as the sulfonic and boronic acid analogs of amino acids that are biologically functional equivalents are also useful in the compounds of the present invention and include compounds having one or more amide linkages optionally replaced by an isostere. In the context of the present invention, for example, —CONH— may be replaced by —CH₂NH—, —NHCO—, —SO₂NH—, —CH₂O—, —CH₂CH₂—, —CH₂S—, —CH₂SO—, —CH—CH— (cis or trans), —COCH₂—, —CH(OH)CH₂— and 1,5-disubstituted tetrazole such that the radicals linked by these isosteres would be held in similar orientations to radicals linked by —CONH—. One or more residues in the peptide reagents described herein may comprise peptoids.

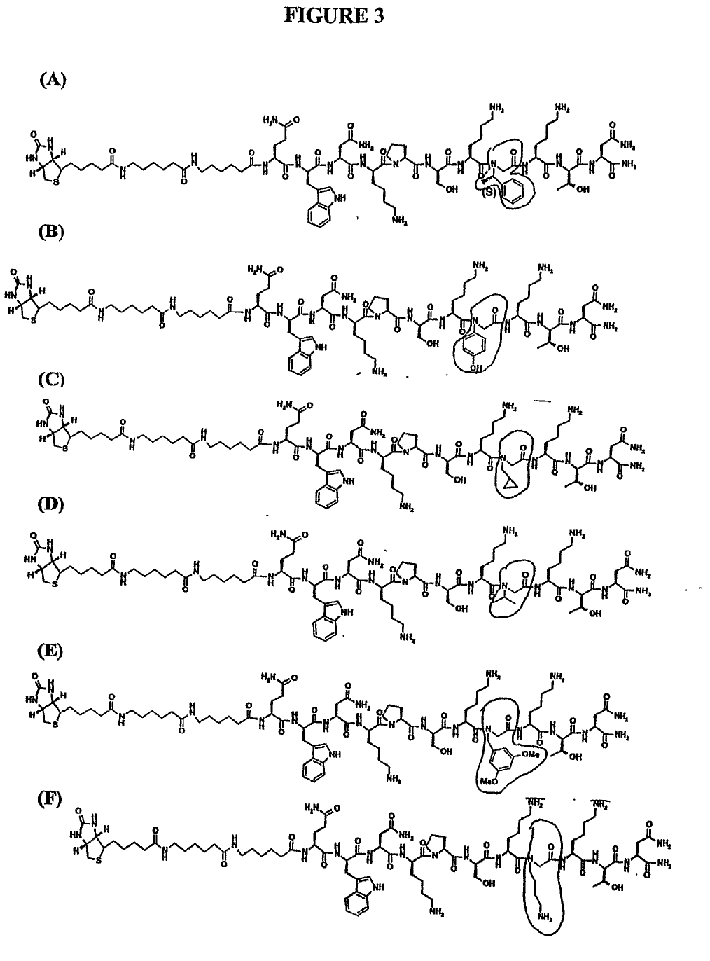

Thus, the peptide reagents also may comprise one or more N-substituted glycine residues (peptides having one or more N-substituted glycine residues may be referred to as “peptoids”). For example, in certain embodiments, one or more proline residues of any of the peptide reagents described herein are replaced with N-substituted glycine residues. Particular N-substituted glycines that are suitable in this regard include, but are not limited to, N—(S)-(1-phenylethyl)glycine; N-(4-hydroxyphenyl)glycine; N-(cyclopropylmethyl)glycine; N-(isopropyl)glycine; N-(3,5-dimethoxybenzyl)glycine; and N-butylglycine. (e.g., FIG. 3). Other N-substituted glycines may also be suitable to replace one or more amino acid residues in the peptide reagents sequences described herein. For a general review of these and other amino acid analogs and peptidomimetics see, Nguyen et al. (2000) Chem Biol. 7(7):463-473; Spatola, A. F., in Chemistry and Biochemistry of Amino Acids, Peptides and Proteins, B. Weinstein, eds., Marcel Dekker, New York, p. 267 (1983). See also, Spatola, A. F., Peptide Backbone Modifications (general review), Vega Data, Vol. 1, Issue 3, (March 1983); Morley, Trends Pharm Sci (general review), pp. 463-468 (1980); Hudson, D. et al., Int J Pept Prot Res, 14:177-185 (1979) (—CH₂NH—, CH₂CH₂—); Spatola et al., Life Sci, 38:1243-1249 (1986) (—CH₂—S); Hann J. Chem. Soc. Perkin Trans. I, 307-314 (1982) (—CH—CH—, cis and trans); Almquist et al., J Med Chem, 23:1392-1398 (1980) (—COCH₂—); Jennings-White et al., Tetrahedron Lett, 23:2533 (1982) (—COCH₂—); Szelke et al., European Appln. EP 45665 CA: 97:39405 (1982) (—CH(OH)CH₂—); Holladay et al., Tetrahedron Lett, 24:4401-4404 (1983) (—C(OH)CH₂—); and Hruby, Life Sci, 31:189-199 (1982) (—CH₂—S—); each of which is incorporated herein by reference. The C-terminal carboxylic acid can be replaced by a boronic acid —B(OH)₂ or boronic ester —B(OR)₂ or other such boronic acid derivative as disclosed in U.S. Pat. No. 5,288,707, incorporated herein by reference.

The peptide reagents described herein may comprise monomers, multimers, cyclized molecules, branched molecules, linkers and the like. Multimers (i.e., dimers, trimers and the like) of any of the sequences described herein or biologically functional equivalents thereof are also contemplated. The multimer can be a homomultimer, i.e., composed of identical monomers, e.g., each monomer is the same peptide sequence. Alternatively, the multimer can be a heteromultimer, by which is meant that not all the monomers making up the multimer are identical.

Multimers can be formed by the direct attachment of the monomers to each other or to substrate, including, for example, multiple antigenic peptides (MAPS) (e.g., symmetric MAPS), peptides attached to polymer scaffolds, e.g., a PEG scaffold and/or peptides linked in tandem with or without spacer units.

Alternatively, linking groups can be added to the monomeric sequences to join the monomers together and form a multimer. Non-limiting examples of multimers using linking groups include tandem repeats using glycine linkers; MAPS attached via a linker to a substrate and/or linearly linked peptides attached via linkers to a scaffold. Linking groups may involve using bifunctional spacer units (either homobifunctional or heterobifunctional) as are known to one of skill in the art. By way of example and not limitation, many methods for incorporating such spacer units in linking peptides together using reagents such as succinimidyl-4-(p-maleimidomethyl)cyclohexane-1-carboxylate (SMCC), succinimidyl-4-(p-maleimidophenyl)butyrate and the like are described in the Pierce Immunotechnology Handbook (Pierce Chemical Co., Rockville, Ill.) and are also available from Sigma Chemical Co. (St. Louis, Mo.) and Aldrich Chemical Co. (Milwaukee, Wis.) and described in “Comprehensive Organic Transformations”, VCK-Verlagsgesellschaft, Weinheim/Germany (1989). One example of a linking group which may be used to link the monomeric sequences together is —Y₁—F—Y₂ where Y₁ and Y₂ are identical or different and are alkylene groups of 0-20, preferably 0-8, more preferably 0-3 carbon atoms, and F is one or more functional groups such as —O—, —S—, —S—S—, —C(O)—O—, —NR—, —C(O)—NR—, —NR—C(O)—O—, —NR—C(O)—NR—, —NR—C(S)—NR—, —NR—C(S)—O—. Y₁ and Y₂ may be optionally substituted with hydroxy, alkoxy, hydroxyalkyl, alkoxyalkyl, amino, carboxyl, carboxyalkyl and the like. It will be understood that any appropriate atom of the monomer can be attached to the linking group.

Further, the peptide reagents of the invention may be linear, branched or cyclized. Monomer units can be cyclized or may be linked together to provide the multimers in a linear or branched fashion, in the form of a ring (for example, a macrocycle), in the form of a star (dendrimers) or in the form of a ball (e.g., fullerenes). Skilled artisans will readily recognize a multitude of polymers that can be formed from the monomeric sequences disclosed herein. In certain embodiments, the multimer is a cyclic dimer. Using the same terminology as above, the dimer can be a homodimer or a heterodimer.

Cyclic forms, whether monomer or multimer, can be made by any of the linkages described above, such as but not limited to, for example: (1) cyclizing the N-terminal amine with the C-terminal carboxylic acid either via direct amide bond formation between the nitrogen and the C-terminal carbonyl, or via the intermediacy of spacer group such as for example by condensation with an epsilon-amino carboxylic acid; (2) cyclizing via the formation of a bond between the side chains of two residues, e.g., by forming a amide bond between an aspartate or glutamate side chain and a lysine side chain, or by disulfide bond formation between two cysteine side chains or between a penicillamine and cysteine side chain or between two penicillamine side chains; (3) cyclizing via formation of an amide bond between a side chain (e.g., aspartate or lysine) and either the N-terminal amine or the C-terminal carboxyl respectively; and/or (4) linking two side chains via the intermediacy of a short carbon spacer group.

Preferably, the peptide reagents described herein are not pathogenic and/or infectious.

The peptide reagents of the invention can be anywhere from 3 to about 100 residues long (or any value therebetween) or even longer, preferably from about 4 to 75 residues (or any value therebetween), preferably from about 5 to about 63 residues (or any value therebetween), and even more preferably from about 8 to about 30 residues (or any value therebetween), and most preferably the peptide reagent will be 10, 11, 12, 13, 14, 15, 16, 17, 18, 19, 20, 21, 22, 23, 24, 25, 26, 27, 28, 29 or 30 residues.

Non-limiting examples of peptide reagents useful in the compositions and methods described herein are derived from sequences shown in Table 1 and in Table 4. Peptide reagents in the tables are represented by conventional one letter amino acid codes and are depicted with their N-terminus at the left and C-terminus at the right. Amino acids in square brackets indicate alternative residues that can be used at that position in different peptide reagents. Round brackets indicate the residue(s) may be present or absent from the peptide reagent. Any proline residue may be substituted with N-substituted glycine residues to form peptoids. Any of the sequences in the tables may optionally include Gly linkers (Gₙ where n=1, 2, 3, or 4) at the N- and/or C-terminal.

TABLE 1

| | |
|---|---|
| | SEQ |
| | ID |
| Peptide sequence | NO |

| | |
|---|---|
| KKRPK | 12 |
| MANLGCWMLVLFVATWSDLGLC | 13 |
| (GGG)QWNKPSKPKTN | 14 |
| QWNKPSKPKTNMKHV | 15 |
| NQNN[N/T]FVHDCVNIT[I/V]K[Q/E]HTVTTTTKGEN | 16 |
| TTKGENFTETD | 17 |
| GENFTETD | 18 |
| GENFTETD[V/I]K[M/I]MERVVEQMC[I/V]TQY[E/Q]ESQAY | 19 |
| Y[Q/D](G)(R)R[G/S][S/A]S | |
| NQNN[N/T]FVHDCVNIT[I/V]K[Q/E]HTVTTTTKGENFTETD | 20 |
| [V/I]K[M/I]MERVVEQMC[I/V]TQY[E/Q]ESQAYY[Q/D](G) | |
| (R)R[G/S][S/A]S | |
| [A/V/T/M][V/I]LFSSPPVILLISFLIFL[I/M]VG | 21 |
| G[N/S]D[W/Y]EDRYYRENM[H/Y]RYPNQVYYRP[M/V]D[Q/E/ | 22 |
| R]Y[S/N]NQN[N/T]FVH | |
| N[N/T]FVHDCVNIT[I/V]K[Q/E]HTVTTTTK | 23 |
| VYYR | 24 |
| RYPNQVYYRP[M/V]D[Q/E/R] | 25 |
| KKRPKPGG(G)WNTGGSRYPGQGSPGGNRYPPQGG | 26 |
| WNTGGSRYPGQGSPGGNRYPPQGG(G) | 27 |
| WNTGGSRYPGQGSPGGNRYPPQGG(G)[G/T]WGQPHGG | 28 |
| GGWGQGGGTHSQWNKPSKPKTN | 29 |
| GGTHSQWNKPSKPKTN | 30 |
| WNTGGSRYPGQGSPGGNRYPPQGG(G)[G/T]WGQPHGGGWGQPHG | 31 |
| GGWGQPHGG | |
| GQPHGGGW | 32 |
| RPIIHGGSDYEDRYYRENMHR | 33 |
| RPMIHFGNDWEDRYYRENMYR | 34 |
| (GGGG)C(GG)GGWGQGGGNQWNKPSKPKTNLKHV(GGGG)C | 35 |
| (GGGG)GGWGQGGGTHNQWNKPSKPKTNLKHV | 36 |
| GGWGQGGGTHNQWNKPSKPKTNLKHV(GGGG) | 37 |
| [M/L]KH[M/V] | 38 |
| KPKTN[M/L]KH[M/V] | 39 |
| C(GG)GGWGQGGGTHNQWNKPSKPKTNLKHV(GGGG)C | 40 |
| SRPIIHFGSDYEDRYYRENMHRYPN | 41 |
| PMIHFGNDWEDRYYRENMYRPVD | 42 |
| AGAAAAGAVVGGLGGYMLGSAM | 43 |
| RPMIHFGNDWEDRYYRENMYR(GGG) | 44 |
| GGGRPMIHFGNDWEDRYYRENMYRGG | 45 |
| (GG)C(GGG)RPMIHFGNDWEDRYYRENMYR(GGG)C | 46 |
| AGAAAAGAVVGGLGG | 47 |
| GGLGG | 48 |
| LGS | 49 |
| QWNKPSKPKTN(GGG) | 50 |
| QWNKPSKPKTN(GGG)QWNKPSKPKTN | 51 |
| QWNKPSKPKTNLKHTV(GGG) | 52 |
| GGWGQGGGTHNQWNKPSKPKTN | 53 |
| GGTHNQWNKPSKPKTN | 54 |
| (GGG)AGAAAAGAVVGGLGGYMLGSAM | 55 |
| (GGG)AGAAAAGAVVGGLGG | 56 |
| (KKK)AGAAAAGAVVGGLGGYMLGSAM | 57 |
| YMLGSAM[S/N]R | 58 |
| [S/N]RP[M/I/L][I/L]H | 59 |
| YMLGSAM[S/N]RP[M/I/L][I/L]H | 60 |
| YMLGSAM[S/N]RP[M/I/L][I/L]HFG[N/S]D | 61 |
| [W/Y]EDRYYRENM[H/Y]RYPNQVYYRP[M/V]D[Q/E/R]Y | 62 |
| [W/Y]EDRYRENM[H/Y]RYPNQVYYRP[M/V]D[Q/E/R]Y[S/N] | 63 |
| NQN[N/T] | |
| D[Q/E/R]Y[S/N]NQN[N/T] | 64 |
| (KKK)AGAAAAGAVVGGLGG | 65 |
| (GGG)KKRPKPGGWNTGGSRYPGQGS | 66 |
| (GGG)KKRPKPGGWNTGG | 67 |
| (GGG)KKRPKPGG | 68 |
| PHGGGWGQHGGSWGQPHGGSWGQ | 69 |
| PHGGGWGQPHGGSWGQ | 70 |
| PHGGGWGQ | 71 |
| (GGG)KKRPKPGGGKKRPKPGG | 72 |
| (GGG)GPKRKGPK | 73 |
| (GGG)WNTGGSRYPGQGS | 74 |
| (GGG)WNKPSKPKT | 75 |
| (GGG)RPMIHFGNDWEDRYYRENMYR(GG)C | 76 |
| QWNKPSKPKTNLKHV(GGG) | 77 |
| (GGG)AGAAAAGAVVGGLGGYMLGSAM | 78 |
| (GGG)NKPSKPK | 79 |
| (GGG)KPSKPK | 80 |
| (GGG)KKRPKPGGGQWNKPSKPKTN | 81 |
| KKKAGAAAAGAVVGGLGGYMLGSANWDD | 82 |
| DDDAGAAAAGAVVGGLGGYMLGSAM | 83 |
| KKKAGAAAAGAVVGGLGGYMLGSAMKKK | 84 |
| (GGG)KKKKKKKK | 85 |
| DDDAGAAAAGAVVGGLGGYMLGSAMDDD | 86 |
| (GGG)NNKQSPWPTKK | 87 |
| DKDKGGVGALAGAAVAAGGDKDK | 88 |
| (GGG)QANKPSKPKTN | 89 |
| (GGG)QWNKASKPKTN | 90 |
| (GGG)QWNKPSKAKTN | 91 |
| (GGG)QWNAPSKPKTN | 92 |
| (GGG)QWNKPSAPKTN | 93 |
| (GGG)QWNKPSKPATN | 94 |
| (GGG)QWNKASKAKTN | 95 |
| (GGG)KKRAKPGG | 96 |
| (GGG)KKRPKAGG | 97 |
| (GGG)KKRAKAGG | 98 |
| (GGG)QWNKASKPKTN | 99 |
| (GGG)QWAKPSKPKTN | 100 |
| (GGG)QWNKPAKPKTN | 101 |
| (GGG)QWNKPSKPKAN | 102 |
| (GGG)QWNKPSKPKTA | 103 |
| (GGG)AKRPKPGG | 104 |
| (GGG)KARPKPGG | 105 |
| (GGG)KKAPKPGG | 106 |
| (GGG)KKRPAPGG | 107 |
| (GGG)KKAPKAGG | 108 |
| (GGG)KKRPKPGGGWNTGG | 127 |
| QWNKPSKPKTNGGGQWNKPSKPKTNGGGQWNKPSKPKTN | 128 |
| ((QWNKPSKPKTN))2K | 133 |
| 4-branchMAPS-GGGKKRPKPGGWNTGGG | 134 |
| 8-branchMAPS-GGGKKRPKPGGWNTGGG | 135 |
| KKKAGAAAAGAVVGGLGG-CONH2 | 136 |
| DLGLCKKRPKPGGXWNTGG | 137 |
| DLGLCKKRPKPGGXWNTG | 138 |
| DLGLCKKRPKPGGXWNT | 139 |
| DLGLCKKRPKPGGXWN | 140 |
| DLGLCKKRPKPGGXW | 141 |
| DLGLCKKRPKPGGX | 142 |
| LGLCKKRPKPGGXWNTG | 143 |
| LCLCKKRPKPGGXWNT | 144 |
| LGLCKKRPKPGGXWN | 145 |
| LGLCKKRPKPGGXW | 146 |
| LGLCKKRPKPGGX | 147 |
| GLCKKRPKPGGXWNTGG | 148 |
| GLCKKRPKPGGXWNTG | 149 |
| GLCKKRPKPGGXWNT | 150 |
| GLCKKRPKPGGXWN | 151 |
| GLCKKRPKPGGXW | 152 |
| GLCKKRPKPGGX | 153 |
| LCKKRPKPGGXWNTGG | 154 |
| LCKKRPKPGGXWNTG | 155 |
| LCKKRPKPGGXWNT | 156 |
| LCKKRPKPGGXWN | 157 |
| LCKKRPKPGGXW | 158. |
| LCKRRPKPGGX | 159 |
| CKKRPKPGGXWNTGG | 160 |
| CKKRPKPGGXWNTG | 161 |
| CKKRPKPGGXWNT | 162 |
| CKKRPKPGGXWN | 163 |
| CKKRPKPGGXW | 164 |
| CKKRPKPGGX | 165 |
| KKRPKPGGXWNTGG | 166 |
| KKRPKPGGXWNTG | 167 |
| KKRPKPGGXWNT | 168 |
| KKRPKPGGXWN | 169 |
| KKRPKPGGXW | 170 |
| KKRPKPGGX | 171 |
| DVGLCKKRPKPGGXWNTGG | 172 |
| DVGLCKKRPKPGGXWNTG | 173 |
| DVGLCKKRPKPGGXWNT | 174 |
| DVGLCKKRPKPGGXWN | 175 |
| DVGLCKKRPKPGGXW | 176 |
| DVGLCKKRPKPGGX | 177 |
| VGLCKKRPKPGGXWNTG | 178 |
| VGLCKKRPKPGGXWNT | 179 |
| VGLCKKRPKPGGXWN | 180 |
| VGLCKKRPKPGGXW | 181 |
| VGLCKKRPKPGGX | 182 |
| THSQWNXPSKPKTNMKHM | 183 |
| THSQWNKPSKPKTNMKH | 184 |
| THSQWNKPSKPKTNMK | 185 |
| THSQWNKPSKPKTNM | 186 |
| THSQWN7KPSKPKTN | 187 |
| HSQWNKPSKPKTNMKHM | 188 |
| HSQWNKPSKPKTNMKH | 189 |
| HSQWNXPSKPKTNMK | 190 |
| HSQWNKPSKPKTNM | 191 |
| HSQWNKPSKPKTN | 192 |
| SQWNKPSKPKTNMKHM | 193 |
| SQWNKPSKPKTNMKH | 194 |
| SQWNXPSKPKTNMK | 195 |
| SQWNKPSKPKTNM | 196 |
| SQWNKPSKPKTN | 197 |
| QWNKPSKPKTNMKHM | 198 |
| QWNKPSKPKTNMKH | 199 |
| QWNKPSKPKTNMK | 200 |
| QWNKPSKPKTNM | 201 |
| THSQWNKPSKPKTNMKHV | 202 |
| HSQWNKPSKPKTNMKHV | 203 |
| SQWNKPSKPKTNMKHV | 204 |
| QWNKPSKPKTNMKHV | 205 |
| THGQWNKPSKPKTNMKHM | 206 |
| THGQWNKPSKPKTNMKH | 207 |
| THGQWNKPSKPKTNMK | 208 |
| THGQWNKPSKPKTNM | 209 |
| THGQWNXPSKPKTN | 210 |
| HGQWNKPSKPKTNMKHM | 211 |
| HGQWNKPSKPKTNMKH | 212 |
| HGQWNKPSKPKTNMK | 213 |
| HGQWNKPSKPKTNM | 214 |
| HGQWNKPSKPKTN | 215 |
| GQWNKPSKPKTNMKHM | 216 |
| GQWNKPSKPKTNMKH | 217 |
| GQWNKPSKPKTNMK | 218 |
| GQWNKPSKPKTNM | 219 |
| GQWNKPSKPKTN | 220 |
| THGQWNKPSKPKTNMKHV | 221 |
| HGQWNKPSKPKTNMKHV | 222 |
| GQWNKPSKPKTNMKHV | 223 |
| THNQWNXPSKPKTNMKHM | 224 |
| THNQWNKPSKPKTNMKH | 225 |
| THNQWNKPSKPKTNMK | 226 |
| THNQWNKPSKPKTNM | 227 |
| THNQWNKPSKPKTN | 228 |
| HNQWNKPSKPKTNMKHM | 229 |
| HNQWNKPSKPKTNMKH | 230 |
| HNQWNKPSKPKTNMK | 231 |
| HNQWNKPSKPKTNM | 232 |
| HNQWNKPSKPKTN | 233 |
| NQWNKPSKPKTNMKHM | 234 |
| NQWNKPSKPKTNMKH | 235 |
| NQWNKPSKPKTNMK | 236 |
| NQWNKPSKPKTNM | 237 |
| NQWNKPSKPKTN | 238 |
| THNQWNKPSKPKTNMKHV | 239 |
| HNQWNKPSKPKTNMKHV | 240 |
| NQWNKPSKPKTNMKHV | 241 |
| PHGGGWGQPHGGGWGQPHGGGWGQ | 242 |
| GGWGQGGGTHSQWNKPSKPKTNMKHM | 243 |
| QWNKPSKPKTNMKHMGGGQWNKPSKPKTNMKHM | 244 |
| GGWGQGGGTH[N/S]QWNKPSKPKTN[L/M]KH[V/M]/M](GGGG) | 245 |
| PHGGGWGQHG[G/S]SWGQPHGG[G/S]WGQ | 246 |
| QWNKPSKPKTN[L/M]KH(V/M](GGG) | 247 |
| 4-branchMAPS-(GGG)QWNKPSKPKTN(GGG) | 259 |
| 8-branchMAPS-(GGG)KKRPKPGGWNT(GGG) | 260 |

In one aspect, the peptide reagent of the invention includes each of the peptides disclosed herein and derivatives (as described herein) thereof. The invention thus includes a peptide reagent derived from a peptide of any of the sequences shown in SEQ ID NO: 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, or 260 and analogs (e.g., substitution of one or more proline with a N-substituted glycine) and derivatives thereof.

The invention preferably includes a peptide reagent derived from a peptide of SEQ ID NO: 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 72, 74, 76, 77, 78, 81, 82, 84, 89, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 249, 250, 251, 252, 253, 254, 255, 256, 257, 258, 259, or 260 and analogs (e.g., substitution of one or more proline with a N-substituted glycine) and derivatives thereof.

In certain preferred embodiments, the peptide reagents specifically bind to pathogenic prions, for example peptide reagents derived from peptides of SEQ ID NOs: 66, 67, 68, 72, 81, 96, 97, 98, 107, 108, 119, 120, 121, 122, 123, 124, 125, 126, 127, 14, 35, 36, 37, 40, 50, 51, 77, 89, 100, 101, 109, 110, 111, 112, 113, 114, 115, 116, 117, 118, 128, 129, 130, 131, 132, 56, 57, 65, 82, 84, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 249, 250, 251, 252, 253, 254, 255, 256, 257, 258, 259, or 260, and analogs (e.g., substitution of one or more proline with a N-substituted glycine) and derivatives thereof.

As described above, the peptide reagents described herein may include one or more substitutions, additions, and/or mutations. For example, one or more residues may be replaced in the peptide reagents with other residues, for example alanine residues or with an amino acid analog or N-substituted glycine residue in order to make a peptoid (see, e.g., Nguyen et al. (2000) Chem Biol. 7(7):463-473).

Furthermore, the peptide reagents described herein may also include additional peptide or non-peptide components. Non-limiting examples of additional peptide components include spacer residues, for example two or more glycine (natural or derivatized) residues or aminohexanoic acid linkers on one or both ends or residues that may aid in solubilizing the peptide reagents, for example acidic residues such as aspartic acid (Asp or D) as depicted for example in SEQ ID NOs:83, 86. In certain embodiments, for example, the peptide reagents are synthesized as multiple antigenic peptides (MAPs). Typically, multiple copies of the peptide reagents (e.g., 2-10 copies) are synthesized directly onto a MAP carrier such as a branched lysine or other MAP carrier core. See, e.g., Wu et al. (2001) J Am Chem Soc. 2001 123(28):6778-84; Spetzler et al. (1995) Int J Pept Protein Res. 45(1):78-85.

Non-limiting examples of non-peptide components (e.g., chemical moieties) that may be included in the peptide reagents described herein include, one or more detectable labels, tags (e.g., biotin, His-Tags, oligonucleotides), dyes, members of a binding pair, and the like, at either terminus or internal to the peptide reagent. The non-peptide components may also be attached (e.g., via covalent attachment of one or more labels), directly or through a spacer (e.g., an amide group), to position(s) on the compound that are predicted by quantitative structure-activity data and/or molecular modeling to be non-interfering. Peptide reagents as described herein may also include prion-specific chemical moieties such as amyloid-specific dyes (e.g., Congo Red, Thioflavin, etc.). Derivatization (e.g., labeling, cyclizing, attachment of chemical moieties, etc.) of compounds should not substantially interfere with (and may even enhance) the binding properties, biological function and/or pharmacological activity of the peptide reagent.

The peptide reagents of the invention will typically have at least about 50% sequence identity to prion protein fragments or to the peptide sequences set forth herein. Preferably, the peptide reagents will have at least 70% sequence identity: more preferably at least 75, 80, 85, 90, 91, 92, 93, 94, 95, 96, 97, 98, 99, or 100% sequence identity to prion protein fragments or to the peptide sequences set forth herein.

The peptide reagents as described herein interact preferentially with the pathogenic forms and, accordingly, are useful in a wide range of isolation, purification, detection, diagnostic and therapeutic applications. For example, in embodiments in which the peptide reagent interacts preferentially with pathogenic forms, the peptide reagents themselves can be used to detect pathogenic forms in a sample, such as a blood, nervous system tissue (brain, spinal cord, CSF, etc) or other tissue or organ sample. The peptide reagents are also useful to diagnose the presence of disease associated with the pathogenic forms, to isolate the pathogenic forms and to decontaminate samples by removing the pathogenic forms.

The interaction of the peptide reagents with prion proteins can be tested using any known binding assay, for example standard immuno assays such as ELISAs, Western blots and the like.

Thus, non-limiting examples of methods of evaluating binding specificity and/or affinity of the peptide reagents described herein include standard Western and Far-Western Blotting procedures; labeled peptides; ELISA-like assays; and/or cell based assays. Western blots, for example, typically employ a tagged primary antibody that detects denatured prion protein from an SDS-PAGE gel, on samples obtained from a “pull-down” assay (as described herein), which has been electroblotted onto nitrocellulose or PVDF. Antibodies that recognize denatured prion protein have been described (described, inter alia, in Peretz et al. 1997 J. Mol. Biol. 273: 614; Peretz et al. 2001 Nature 412:739; Williamson et al. 1998 J. Virol. 72:9413; U.S. Pat. No. 6,765,088; U.S. Pat. No. 6,537,548) and some are commercially available. Other prion-binding molecules have been described e.g., motif-grafted hybrid polypeptides (see, WO03/085086), certain cationic or anionic polymers (see, WO03/073106), certain peptides that are “propagation catalysts” (see, WO02/0974444) and plasminogen. The primary antibody is then detected (and/or amplified) with a probe for the tag (e.g., streptavidin-conjugated alkaline phosphatase, horseradish peroxidase, ECL reagent, and/or amplifiable oligonucleotides). Binding can also be evaluated using detection reagents such as a peptide with an affinity tag (e.g., biotin) that is labeled and amplified with a probe for the affinity tag (e.g., streptavidin-conjugated alkaline phosphatase, horseradish peroxidase, ECL reagent, or amplifiable oligonucleotides). In addition, microtitre plate procedures similar to sandwich ELISA may be used, for example, a prion-specific peptide reagent as described herein is used to immobilize prion protein(s) on a solid support (e.g., well of a microtiter plate, bead, etc.) and an additional detection reagent which could include, but is not limited to, another prion-specific peptide reagent with an affinity and/or detection label such as a conjugated alkaline phosphatase, horseradish peroxidase, ECL reagent, or amplifiable oligonucleotides. Cell based assays can also be employed, for example, where the prion protein is detected directly on individual cells (e.g., using a fluorescently labeled prion-specific peptide reagent that enables fluorescence based cell sorting, counting, or detection of the specifically labeled cells).

### III.B. PEPTIDE REAGENT PRODUCTION

The peptide reagents of the present invention can be produced in any number of ways, all of which are well known in the art.

In one embodiment, in which the peptide reagent is, in whole or in part, a genetically encoded peptide, the peptide can be generated using recombinant techniques, well known in the art. One of skill in the art could readily determine nucleotide sequences that encode the desired peptide using standard methodology and the teachings herein. Once isolated, the recombinant peptide, optionally, can be modified to include non-genetically encoded components (e.g., detectable labels, binding pair members, etc.) as described herein and as well-known in the art, to produce the peptide reagents.

Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations. See, e.g., Sambrook et al., supra, for a description of techniques used to obtain and isolate DNA.

The sequences encoding the peptide can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone sequences encoding polypeptides useful in the claimed peptide reagents that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci. USA (1982) 79:6409.

Once coding sequences have been isolated and/or synthesized, they can be cloned into any suitable vector or replicon for expression. (See, also, Examples). As will be apparent from the teachings herein, a wide variety of vectors encoding modified polypeptides can be generated by creating expression constructs which operably link, in various combinations, polynucleotides encoding polypeptides having deletions or mutations therein.

Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra. Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit).

Plant expression systems can also be used to produce the peptide reagents described herein. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

Viral systems, such as a vaccinia based infection/transfection system, as described in Tomei et al., J. Virol. (1993) 67:4017-4026 and Selby et al., J. Gen. Virol. (1993) 74:1103-1113, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. With the present invention, both the naturally occurring signal peptides or heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honey bee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector that already contains the control sequences and an appropriate restriction site.

In some cases it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the proteins of the present invention are produced by growing host cells transformed by an expression vector described above under conditions whereby the protein of interest is expressed. The selection of the appropriate growth conditions is within the skill of the art.

In one embodiment, the transformed cells secrete the polypeptide product into the surrounding media. Certain regulatory sequences can be included in the vector to enhance secretion of the protein product, for example using a tissue plasminogen activator (TPA) leader sequence, an interferon (γ or α) signal sequence or other signal peptide sequences from known secretory proteins. The secreted polypeptide product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the recombinant polypeptides substantially intact. Intracellular proteins can also be obtained by removing components from the cell wall or membrane, e.g., by the use of detergents or organic solvents, such that leakage of the polypeptides occurs. Such methods are known to those of skill in the art and are described in, e.g., Protein Purification Applications: A Practical Approach, (E. L. V. Harris and S. Angal, Eds., 1990).

For example, methods of disrupting cells for use with the present invention include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced polypeptides are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining the intracellular polypeptides of the present invention involves affinity purification, such as by immunoaffinity chromatography using antibodies (e.g., previously generated antibodies), or by lectin affinity chromatography. Particularly preferred lectin resins are those that recognize mannose moieties such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (NPA) and Allium ursinum agglutinin (AUA). The choice of a suitable affinity resin is within the skill in the art. After affinity purification, the polypeptides can be further purified using conventional techniques well known in the art, such as by any of the techniques described above.

Peptide reagents can be conveniently synthesized chemically, for example by any of several techniques that are known to those skilled in the peptide art. In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis. These methods are typically used for relatively small polypeptides, i.e., up to about 50-100 amino acids in length, but are also applicable to larger polypeptides.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

Synthesis of peptoid containing polymers can be carried out according to, e.g., U.S. Pat. Nos. 5,877,278; 6,033,631; Simon et al. (1992) Proc. Natl. Acad. Sci. USA 89:9367.

The peptide reagent of the present invention can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

### IV. ISOLATION

The peptide reagents described herein can be used to isolate PrP^{Sc} in its native conformation or in denatured form. Previously, isolation of native PrP^{Sc} was a difficult and cumbersome procedure, requiring sonication, centrifugation, solubilization in detergent, and/or incorporation into liposomes. See, e.g., Gabizon et al. (1987) Proc. Nat'l Acad. Sci. USA 84:4017-4021; Peretz et al. (1997) J. Mol. Biol. 273:614-622. In addition, previous procedures digested the brain homogenates with Proteinase K, in order to exclude PrP^{C}. However, since the N-terminus (23-90) is PK-sensitive, PK treatment gives a truncated proteinase resistant fragment of PrP^{Sc} spanning residues 90-231 and full-length native PrP^{Sc} is not readily isolated.

The present invention thus provides a method for isolating pathogenic prion protein in the native conformation comprising the steps of: (a) contacting a sample containing a pathogenic prion protein with a peptide reagent as described herein under conditions that allow the binding of the pathogenic prion protein, if present in said sample, to the peptide reagent to form a first complex; and (b) removing unbound sample materials.

The invention also provides a method for isolating a pathogenic prion protein in the native conformation comprising the steps of: (a) contacting a sample containing a pathogenic prion protein with a peptide reagent as described herein under conditions that allow the binding of the pathogenic prion protein, if present in said sample, to the peptide reagent to form a first complex; (b) removing unbound sample materials; and (c) dissociating the pathogenic prion protein from the peptide reagent under non-denaturing conditions.

The invention also provides a method of isolating a pathogenic prion protein in the native conformation, the method comprising the steps of: (a) providing a solid support comprising a first peptide reagent; (b) contacting the solid support with a sample under conditions which allow pathogenic prions, when present in the sample, to bind to the first peptide reagent; (c) removing unbound sample materials; and (d) dissociating the pathogenic prions from the solid support under non-denaturing conditions.

Any of the methods of isolating non-denatured pathogenic prions may further comprise the step of separating the dissociated pathogenic prions from the peptide reagent, for example by centrifugation, precipitation, removal of the peptide by magnetic pull down, etc.

Furthermore, in any of the methods of isolating pathogenic prion proteins in their native conformation described herein, the dissociation step can comprise contacting the first complex with a non-denaturing concentration of a salt or a chaotropic agent. The salt may be, for example, NaCl or KCl. The chaotropic agent may be, for example, GdnSCN or GdnHCl. In preferred embodiments, the concentration of salt is between about 0.5M and 2 M, more preferably between about 1.0 M and about 1.5 M, and even more preferably, 1.0 M, 1.1 M, 1.2 M, 1.3 M, or 1.4 M. In preferred embodiments using GdnSCN or GdnHCl, the concentration of chaotropic agent is between about 0.4 M and 2 M, more preferably between about 0.4 M and about 1.0 M, and even more preferably, 0.5 M, 0.6 M, 0.7 M, 0.8 M, 0.9 M or 1.0 M. Alternatively or in addition to use of salts and chaotropic agents, centrifugation and/or precipitation steps may also be used to isolate non-denatured pathogenic prion proteins.

Thus, the methods described herein allow for the efficient and simple isolation of PrP^{Sc}, which in turn facilitates a variety of applications such as antibody production, structural studies, drug screening and the like.

Briefly stated, the methods of isolating PrP^{Sc} as described herein involve contacting the sample containing PrP^{Sc} with peptide reagents as described herein under conditions such that the PrP^{Sc} binds to the peptide reagent(s). The peptide reagent may be bound to a solid support, for example beads, and contacted with a sample suspected of containing PrP^{Sc} under conditions such that the PrP^{Sc} present in the sample binds to the peptide reagent(s). In certain embodiments, the PrP^{Sc} and peptide reagent(s) are contacted at room temperature and at pH between about 6 and about 9.

Isolation of native form PrP^{Sc} using peptide reagents as described herein may further comprise dissociating the PrP^{Sc} from the peptide reagent(s). Dissociation of PrP^{Sc} from the peptide reagent may be accomplished in a variety of ways, so long as the PrP^{sc} remains in its native, non-denatured configuration. The methods generally use a high concentration of a non-denaturing salt (e.g., NaCl or KCl) or a low concentration of a chaotropic agent (e.g., guanidinium isothiocyanate or guanidinium hydrochloride) to dissociate the PrP^{Sc} without denaturing the protein. Alternatively, centrifugation methods can be used to selectively sediment the PrP^{Sc} away from the complex with the peptide reagent.

Thus, in certain embodiments, PrP^{Sc} in its native conformation is dissociated from a peptide reagent as described herein using sodium chloride (NaCl) or potassium chloride (KCl). The molarity of the NaCl or KCl is preferably between about 0.5 M and about 2 M, more preferably between about 1.0 M and about 1.5 M, and even more preferably between about 1.0 M and 1.3 M, including 1.0 M, 1.1 M, 1.2 M and 1.3 M. NaCl is the preferred salt.

In other embodiments, one or more chaotropic salts are used to dissociate native form PrP^{Sc} from the peptide reagent. Non-limiting examples of suitable chaotropic salts include guanidinium isothiocyanate (GdnSCN) and guanidinium hydrochloride (GdnHCl). The concentration of chaotropic salt used will be sufficient to dissociate PrP^{Sc} from the peptide reagent, but not so high as to denature PrP^{Sc}. It has been shown that treatment of PrP^{Sc} with 1 to 2M GdnSCN for 24 hours does not significantly reduce PrP^{Sc} infectivity, while treatment with 3M GdnSCN for 1 hour reduced the majority of infectivity. (Prusiner et al. (1993) Proc. Nat'l Acad. Sci. USA 90:2793-2797). Similarly, treatment with 3M GdnSCN has been shown to denature PrP^{Sc} such that it can be detected with antibodies that recognize PrP^{C} and denatured PrP^{Sc} (e.g., D18). See, e.g., Peretz et al. (1997) J. Mol. Biol. 273(3):614-622; Williamson et al. (1998) J. Virol. 72(11):9413-9418. Accordingly, when GdnSCN or GdnHCl are used to dissociate PrP^{Sc} from the peptide reagents described herein, the molarity is preferably less than about 3M, more preferably between about 0.4 M and about 2M, most preferably between about 0.5M and about 1.0M, including 0.5M, 0.6M, 0.7M, 0.8M, 0.9M and 1.0M. As demonstrated herein, treatment with 0.5 M to about 2M GdnSCN is sufficient to dissociate PrP^{Sc} from the peptide reagents, without denaturing PrP^{Sc}.

In certain embodiments, isolation of PrP^{Sc} may involve one or more purification steps prior to or after dissociation. For example, high-speed centrifugation may be used to concentrate PrP^{Sc}. The centrifugation is typically conducted at high speeds, preferably at least about 10,000G, more preferably at least about 15,000 G and even more preferably at least about 20,000 G.

In still other embodiments, PrP^{Sc} can be purified in non-denaturing conditions by precipitation, for example using a precipitant such as ammonium sulfate (e.g., using about a 10% saturated solution of ammonium sulfate).

One or more of the purification and/or dissociation methods may also be combined, for example, centrifugation in combination with NaCl of Gdn SCN dissociation, precipitation in combination with centrifugation, and/or a combination of precipitation, centrifugation and NaCl/GdnSCN elutions.

### Solid Supports

One or more of the steps of the methods described herein may be conducted in solution (e.g., a liquid medium) or on a solid support. A solid support, for purposes of the invention, can be any material that is an insoluble matrix and can have a rigid or semi-rigid surface to which a molecule of interest (e.g., peptide reagents of the invention, prion proteins, antibodies, etc) can be linked or attached. Exemplary solid supports include, but are not limited to, substrates such as nitrocellulose, polyvinylchloride, polypropylene, polystyrene, latex, polycarbonate, nylon, dextran, chitin, sand, silica, pumice, agarose, cellulose, glass, metal, polyacrylamide, silicon, rubber, polysaccharides, polyvinyl fluoride, diazotized paper, activated beads, magnetically responsive beads, and any materials commonly used for solid phase synthesis, affinity separations, purifications, hybridization reactions, immunoassays and other such applications. The support can be particulate or can be in the form of a continuous surface and includes membranes, mesh, plates, pellets, slides, disks, capillaries, hollow fibers, needles, pins, chips, solid fibers, gels (e.g. silica gels) and beads, (e.g., pore-glass beads, silica gels, polystyrene beads optionally cross-linked with divinylbenzene, grafted co-poly beads, polyacrylamide beads, latex beads, dimethylacrylamide beads optionally crosslinked with N—N′-bis-acryloylethylenediamine, iron oxide magnetic beads, and glass particles coated with a hydrophobic polymer).

If desired, the molecules to be added to the solid support can readily be functionalized to create styrene or acrylate moieties, thus enabling the incorporation of the molecules into polystyrene, polyacrylate or other polymers such as polyimide, polyacrylamide, polyethylene, polyvinyl, polydiacetylene, polyphenylene-vinylene, polypeptide, polysaccharide, polysulfone, polypyrrole, polyimidazole, polythiophene, polyether, epoxies, silica glass, silica gel, siloxane, polyphosphate, hydrogel, agarose, cellulose and the like.

Peptide reagents as described herein can be readily coupled to the solid support using standard techniques. Immobilization to the support may be enhanced by first coupling the peptide reagent to a protein (e.g., when the protein has better solid phase-binding properties). Suitable coupling proteins include, but are not limited to, macromolecules such as serum albumins including bovine serum albumin (BSA), keyhole limpet hemocyanin, immunoglobulin molecules, thyroglobuline, ovalbumin, and other proteins well known to those skilled in the art. Other reagents that can be used to bind molecules to the support include polysaccharides, polylactic acids, polyglycolic acids, polymeric amino acids, amino acid copolymers, and the like. Such molecules and methods of coupling these molecules to proteins, are well known to those of ordinary skill in the art. See, e.g., Brinkley, M. A., (1992) Bioconjugate Chem., 3:2-13; Hashida et al. (1984) J. Appl. Biochem., 6:56-63; and Anjaneyulu and Staros (1987) International J. of Peptide and Protein Res. 30:117-124.

The peptide reagents can be attached to the solid support through the interaction of a binding pair of molecules. Such binding pairs are well known and examples are described elsewhere herein. One member of the binding pair is coupled by techniques described above to the solid support and the other member of the binding pair is attached to the peptide reagent (before, during, or after synthesis). The peptide reagent thus modified can be contacted with the sample and interaction with the pathogenic prion, if present, can occur in solution, after which the solid support can be contacted with the peptide reagent (or peptide-prion complex). Preferred binding pairs for this embodiment include biotin and avidin, and biotin and streptavidin.

In still further embodiments, the invention is directed to solid supports comprising a pathogenic prion-specific peptide reagent. Methods of producing these solid supports are also provided, for example by (a) providing a solid support; and (b) binding thereto one or more pathogenic prion-specific peptide reagents.

The prion-specific peptide reagents may further be used to isolate pathogenic prion proteins using affinity supports. The peptide reagents can be affixed to a solid support by, for example, adsorption, covalent linkage, etc. so that the peptide reagents retain their prion-selective binding activity. Optionally, spacer groups may be included, for example so that the binding site of the peptide reagent remains accessible. The immobilized molecules can then be used to bind the pathogenic prion protein from a biological sample, such as blood, plasma, brain, spinal cord, other tissues. The bound peptide reagents or complexes are recovered from the support by, for example, a change in pH or the pathogenic prion may be dissociated from the complex.

Samples that can be tested according to the invention include any sample amenable to an antibody assay, including samples from nervous system tissue (e.g., brain, spinal cord, CSF, etc.) blood and/or other tissue samples from living or dead subjects. As noted above, in preferred embodiments, the samples are blood, blood product or tissue samples obtained from a living subject.

### V. ANTIBODIES

Antibodies may be raised against the native PrP^{Sc}, which may be isolated as described herein using the peptide reagents.

The anti-prion antibodies may be polyclonal or monoclonal antibody preparations, monospecific antisera, human antibodies, or may be hybrid or chimeric antibodies, such as humanized antibodies, altered antibodies (Fab′)₂ fragments, F(ab) fragments, Fv fragments, single-domain antibodies, dimeric or trimeric antibody fragments or constructs, minibodies, or functional fragments thereof which bind to the antigen in question.

Antibodies are produced using techniques well known to those of skill in the art and disclosed in, for example, U.S. Pat. Nos. 4,011,308; 4,722,890; 4,016,043; 3,876,504; 3,770,380; and 4,372,745. For example, polyclonal antibodies are generated by immunizing a suitable animal, such as a mouse, rat, rabbit, sheep, or goat, with an antigen of interest (e.g., a peptide reagent as described herein). In order to enhance immunogenicity, the antigen can be linked to a carrier prior to immunization. Such carriers are well known to those of ordinary skill in the art. Immunization is generally performed by mixing or emulsifying the antigen in saline, preferably in an adjuvant such as Freund's complete adjuvant, and injecting the mixture or emulsion parenterally (generally subcutaneously or intramuscularly). The animal is generally boosted 2-6 weeks later with one or more injections of the antigen in saline, preferably using Freund's incomplete adjuvant. Antibodies may also be generated by in vitro immunization, using methods known in the art. Polyclonal antiserum is then obtained from the immunized animal.

Monoclonal antibodies are generally prepared using the method of Kohler and Milstein (1975) Nature 256:495-497, or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the antigen. B-cells, expressing membrane-bound immunoglobulin specific for the antigen, will bind to the plate, and are not rinsed away with the rest of the suspension. Resulting B-cells, or all dissociated spleen cells, are then induced to fuse with myeloma cells for form hybridomas, and are cultured in a selective medium (e.g., hypoxanthine, aminopterin, thymidine medium, “HAT”). The resulting hybridomas are plated by limiting dilution, and are assayed for the production of antibodies that bind specifically to the immunizing antigen (and which do not bind to unrelated antigens). The selected monoclonal antibody-secreting hybridomas are then cultured either in vitro (e.g., in tissue culture bottles or hollow fiber reactors), or in vivo (e.g., as ascites in mice).

Humanized and chimeric antibodies are also useful in the invention. Hybrid (chimeric) antibody molecules are generally discussed in Winter et al. (1991) Nature 349: 293-299 and U.S. Pat. No. 4,816,567. Humanized antibody molecules are generally discussed in Riechmann et al. (1988) Nature 332:323-327; Verhoeyan et al. (1988) Science 239:1534-1536; and U.K. Patent Publication No. GB 2,276,169, published 21 Sep. 1994). One approach to engineering a humanized antibody involves cloning recombinant DNA containing the promoter, leader, and variable-region sequences from a mouse antibody gene and the constant-region exons from a human antibody gene to create a mouse-human antibody, a humanized antibody. See generally, Kuby, “Immunology, 3^{rd} Edition”, W.H. Freeman and Company, New York (1998) at page 136.

Antibodies, both monoclonal and polyclonal, which are directed against PrP^{Sc} isolated using these reagents as described herein are particularly useful in diagnosis and therapeutic applications, for example, those antibodies that are neutralizing are useful in passive immunotherapy. Monoclonal antibodies, in particular, may be used to raise anti-idiotype antibodies.

Anti-idiotype antibodies are immunoglobulins that carry an “internal image” of the antigen of the agent against which protection is desired. Techniques for raising anti-idiotype antibodies are known in the art. See, e.g., Grzych (1985), Nature 316:74; MacNamara et al. (1984), Science 226:1325, Uytdehaag et al (1985), J. Immunol. 134:1225. These anti-idiotype antibodies may also be useful for treatment and/or diagnosis of conformational diseases.

Antibody fragments are also included within the scope of the invention. A number of antibody fragments are known in the art that comprise antigen-binding sites capable of exhibiting immunological binding properties of an intact antibody molecule. For example, functional antibody fragments can be produced by cleaving a constant region, not responsible for antigen binding, from the antibody molecule, using e.g., pepsin, to produce F(ab′)₂ fragments. These fragments will contain two antigen binding sites, but lack a portion of the constant region from each of the heavy chains. Similarly, if desired, Fab fragments, comprising a single antigen binding site, can be produced, e.g., by digestion of polyclonal or: monoclonal antibodies with papain. Functional fragments, including only the variable regions of the heavy and light chains, can also be produced, using standard techniques such as recombinant production or preferential proteolytic cleavage of immunoglobulin molecules. These fragments are known as Fᵥ. See, e.g., Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single-chain Fv (“sFv” or scFv”) polypeptide is a covalently linked V_{H}-V_{L} heterodimer that is expressed from a gene fusion including V_{H}- and V_{L}-encoding genes linked by a peptide-encoding linker. Huston et al. (1988) Proc. Nat. Acad. Sci. USA 85:5879-5883. A number of methods have been described to discern and develop chemical structures (linkers) for converting the naturally aggregated, but chemically separated, light and heavy polypeptide chains from an antibody V region into an sFv molecule which will fold into a three dimensional structure substantially similar to the structure of an antigen-binding site. See, e.g., U.S. Pat. Nos. 5,091,513; 5,132,405; and 4,946,778. The sFv molecules may be produced using methods described in the art. See, e.g., Huston et al. (1988) Proc. Nat. Acad. Sci USA 85:5879-5338; U.S. Pat. Nos. 5,091,513; 5,132,405 and 4,946,778. Design criteria include determining the appropriate length to span the distance between the C-terminus of one chain and the N-terminus of the other, wherein the linker is generally formed from small hydrophilic amino acid residues that do not coil or form secondary structures. Such methods have been described in the art. See, e.g., U.S. Pat. Nos. 5,091,513; 5,132,405 and 4,946,778. Suitable linkers generally comprise polypeptide chains of alternating sets of glycine and serine residues, and may include glutamic acid and lysine residues inserted to enhance solubility.

“Mini-antibodies” or “minibodies” will also find use with the present invention. Minibodies are sFv polypeptide chains that include oligomerization domains at their C-termini, separated from the sFv by a hinge region. Pack et al., (1992) Biochem 31:1579-1584. The oligomerization domain comprises self-associating α-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al., (1992) Biochem 31:1579-1584; Cumber et al. (1992) J. Immunology 149B:120-126.

Non-conventional means can also be used to generate and identify antibodies. For example, a phage display library can be screened for antibodies that bind more to pathogenic forms than non-pathogenic forms or vice versa. See generally, Siegel, “Recombinant Monoclonal Antibody Technology”, Transfus. Clin. Biol. (2002) 9(1): 15-22; Sidhu, “Phage Display in Pharmaceutical Biotechnology”, Curr. Opin. Biotechnol. (2000) 11(6):610-616; Sharon, et al., “Recombinant Polyclonal Antibody Libraries”, Comb. Chem. High Throughput Screen (2000) 3(3): 185-196; and Schmitz et al., “Phage Display: A Molecular Tool for the Generation of Antibodies—Review”, Placenta, (2000) 21 SupplA: S106-12.

The specificity of the antibodies of the invention can be tested as described above for peptide reagents. As mentioned above, prions having a pathogenic conformation are generally resistant to certain proteases, such as proteinase K. The same proteases are able to degrade prions in a non-pathogenic conformation. One method of testing the specificity of the antibodies of the present invention is to select a biological sample containing both pathogenic and non-pathogenic prions. The sample can be separated into two equal volumes. Antibodies of the invention can be added adsorbed onto a solid support (as further described below) and used to obtain a quantitative value directly related to the number of antibody-prion binding interactions on the solid support. Protease can be added to the second sample and the same test performed. Because the protease in the second sample will degrade any non-pathogenic prions, any antibody-prion binding interactions in the second sample can be attributed to pathogenic prions. Variations and other assays known in the art can also be used to demonstrate the specificity of the antibodies of the invention.

### VI. SCREENING

Isolated native PrP^{Sc} may be used to screen for any molecules (proteins, antibodies, etc.) that can bind to PrP^{Sc}, either in the native form or denatured (e.g., post-isolation). In this way, potential therapeutics can be identified. Prions reproduce by recruiting PrP^{C} and stimulating its conversion to PrP^{Sc}. This mechanism suggests that compounds specifically binding either PrP isoform may interrupt prion production by inhibiting this folding process. Libraries of different compounds can be screened for binding to PrP^{Sc} in its various forms. PrP^{Sc} can be presented on beads, or any solid phase, like polystyrene, or nitrocellulose. Following washing, compounds that bind can be readily eluted and identified using standard identification protocols. Binding compounds can be tested for their ability to inhibit PrP^{Sc} binding to PrP^{C} in vitro or in vivo.

### EXAMPLES

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Peptide Reagent Production

Peptide fragments of prion proteins were chemically synthesized using standard peptide synthesis techniques, essentially as described in Merrifield (1969) Advan. Enzymol. 32: 221 and Holm and Medal (1989), Multiple column peptide synthesis, p. 208E, Bayer and G. Jung (ed.), Peptides 1988, Walter de Gruyter & Co. Berlin-N.Y. Peptides were purified by HPLC and sequence verified by mass spectroscopy.

In certain cases, the peptides synthesized included additional residues at the N or C terminus, for example GGG residues and/or included one or more amino acid substitutions as compared to wild-type sequences.

### A. Peptoid Substitutions

Peptoid substitutions were also made in the peptide presented in SEQ ID NO:14 (QWNKPSKPKTN, corresponding to residues 97 to 107 of SEQ ID NO:2), SEQ ID NO:67 (KKRPKPGGWNTGG, corresponding to residues 23-36 of SEQ ID NO:2) and SEQ ID NO:68 (KKRPKPGG, corresponding to residues 23-30 of SEQ ID NO:2). In particular, one or more proline residues of these peptides were substituted with various N-substituted peptoids. See, FIG. 3 for peptoids that can be substituted for any proline. Peptoids were prepared and synthesized as described in U.S. Pat. Nos. 5,877,278 and 6,033,631, both of which are incorporated by reference in their entireties herein; Simon et al. (1992) Proc. Natl. Acad. Sci. USA 89:9367.

### B. Multimerization

Certain peptide reagents were also prepared as multimers, for example by preparing tandem repeats (linking multiple copies of a peptide via linkers such as GGG), multiple antigenic peptides (MAPS) and/or linearly-linked peptides.

In particular, MAPS were prepared using standard techniques, essentially as described in Wu et al. (2001) J Am Chem Soc. 2001 123(28):6778-84; Spetzler et al. (1995) Int J Pept Protein Res. 45(1):78-85.

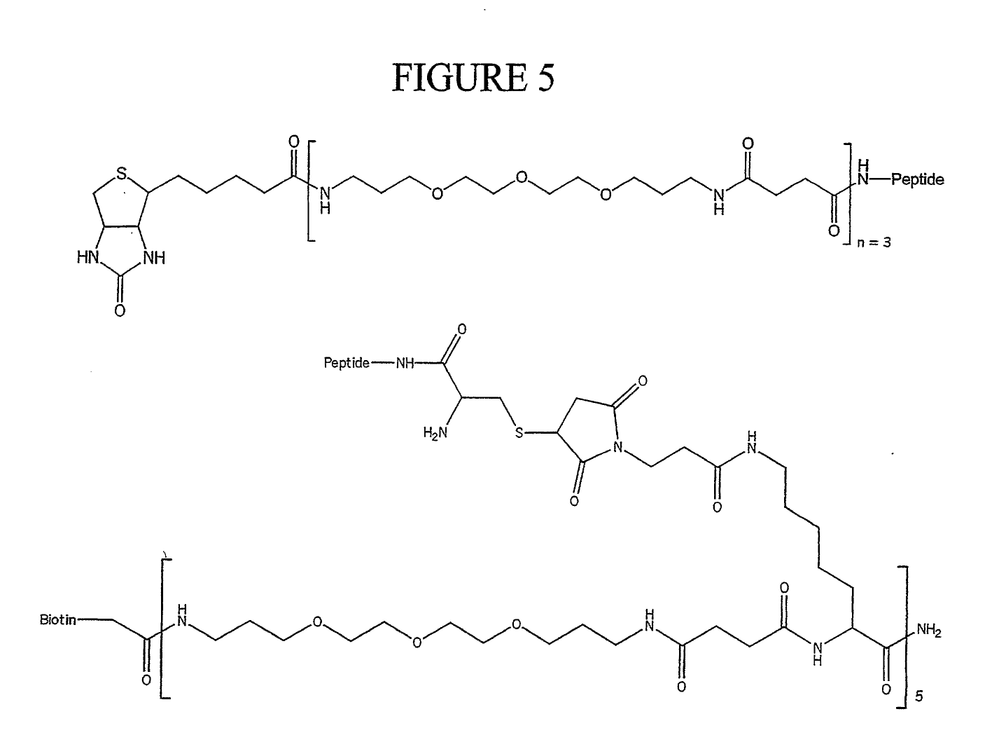

Linear and branched peptides (e.g., PEG linker multimerization) were also prepared using polyethylene glycol (PEG) linkers, using standard techniques. In particular, branched multipeptide PEG scaffolds were created with the following structures: Biotin-PEG-Lys-PEG-Lys-PEG-Lys-PEG-Lys-PEG-Lys (no peptide control) and Biotin-PEG-Lys(Peptide)-PEG-Lys(Peptide)-PEG-Lys(Peptide)-PEG-Lys(Peptide)-PEG-Lys(Peptide). In addition, peptide to Lys linkages were prepared: Lys-epsilon-NH—CO—(CH2)3-Mal-S-Cys-peptide. See, FIG. 5.

### C. Biotinylation

Peptides were biotinylated using standard techniques following synthesis and purification. Biotin was added to the N- or C-terminal of the peptide.

### Example 2

### Binding Assays

### A. Pull-Down

Peptide reagents as described herein were tested for their ability to specifically bind to prion proteins using a magnetic bead pull down assay. For this assay, the peptide reagents were labeled with biotin, which allowed attachment to streptavidin coated magnetic beads.

Brain homogenates are prepared from RML PrP^{Sc+} and PrP^{C+} Balb-c mice. In brief, 5 mL of TBS buffer (50 mM Tris-HCl pH 7.5 and 37.5 mM NaCl) with 1% Tween20 and 1% triton 100 was added to brains weighing ˜0.5 g to produce a 10% homogenate. The brain slurry was dounced until large particles had disappeared. Aliquots of 200 μl were diluted 1:1 in buffer were added to pre-cooled eppendorf tubes and the samples sonicated for several repeats of several seconds each. Samples were centrifuged for 10-15 minutes at 500× and the supernatants removed.

To test the effect of Proteinase K digestion, certain supernatants were divided into two samples and 4 μl of Proteinase K was added to one sample and rotated at 37° C. for 1 hour. Eight microliters of PMSF was added to the proteinase K tubes to stop digestion and the tubes were incubated for a minimum of 1 hour at 4° C.

Homogenates were stored at 4° C. degrees until further use and sonicated again as described above if needed. A 10% w/v PrP^{C+} or PrP^{Sc+} preparation of the brain homogenates was incubated overnight at 4° C. with a biotin-labeled peptide reagent, as follows Tubes containing 400 μl of buffer, 50 μl of extract and 5 μl of biotin-labeled peptide reagent (10 mM stock) were prepared. The tubes were incubated for a minimum of 2 hours at room temperature or overnight at 4° C. on platform rocker.

Following incubation, 50 μl of SA-beads Dynal M280 Streptavidin 112.06) were added and the tubes mixed by vortexing. The tubes were incubated, with rocking (VWR, Rocking platform, Model 100), for 1 hour at room temperature or overnight in at 4° C.

Samples were removed from shaker, placed in magnetic field to collect the magnetic beads with attached peptide reagent and prion and washed 5-6 time using 1 ml assay buffer. Samples were used immediately or stored at −20° C. until Western blotting or ELISA, described below.

### B. Western Blotting

Western blotting analysis was performed as follows. Bead-peptide-prion complexes precipitated as described above were denatured after the final wash by adding 25-30 μl of SDS buffer (Novex Tris-Glycine SDS-Sample Buffer 2×) added to each tube. The tubes were mixed by vortexing until all of the beads were suspended. The tubes were boiled until the tops started to come open, run on a standard SDS-PAGE gel and transferred to a solid membrane for WB analysis.

The membrane was blocked for 30 minutes in 5% Milk/TBS-T [50 ml 1 M Tris pH 7.5; 37.5 ml 4M NaCl, 1-10 mL Tween bring volume to 1 L with milk] at room temperature. Between 10-15 ml of anti-prion polyclonal antibodies, as described in International Application No. PCT/US03/31057, filed Sep. 30, 2003, entitled “Prion Chimeras and Uses Thereof” were added at a 1:50 fold dilution to the membrane and incubated for 1 hour at room temperature. The membrane was washed multiple times in TBS-T. After washing, the secondary antibody (goat anti-rabbit IgG (H+ L) antibody (Pierce) conjugated to alkaline phosphatase (AP) was added at 1:1000 dilution (in TBS-T) and incubated for 20 minutes at room temperature. The membrane was washed multiple times in TBS-T. Alkaline phosphatase precipitating reagent (1-step NBT/BCIP (Pierce) was added and developed until background appeared or signal was apparent.

### C. ELISA

Following the final wash, the bead-peptide complexes described above were denatured with 6M GdnSCN for 10 minutes at room temperature and ELISAs performed on the denatured protein as follows. See, e.g., Peretz et al. (1997) J. Mol. Biol. 273(3):614-622; Ryou et al. (2003) Lab Invest. 83(6):837-43. Denatured PrP^{Sc} was coated onto the plates by incubation with 0.1M NaHCO₃, pH 8.9 (110 μl/well) and the beads removed from the wells by aspiration and washing (3× with 200 μl TBS with 0.05% Tween20).

A 100 μl aliquot of the denatured samples were transferred into duplicate 96-well plates and incubated overnight at 4° C. Following washing, the wells (coated with any PrP^{Sc} in the sample) were blocked with 200 μl of 3% BSA in TBS for 1 hour at 37° C. The blocking solution was then aspirated out of the wells and 100 μl of 0.5 μg/ml solution of primary Fab D18 (Peretz et al. (2001) Nature 412(6848):739-743) in TBS with 1% BSA was added to each well and incubated for 2 hours at 37° C. The wells were then washed 9× with 300 μl of TBC with 0.05% TW2. Goat anti-human antibody conjugated with alkaline phosphatase (AP) was added to each well and the plate incubated for 1 hour at 37° C. After washing (9× with 300 μl of TBC with 0.05% TW2), 100 μl of AP substrate was added to each well, the plates incubated at 37° C. for 0.5 hours and the optical density (OD) of the plates read.

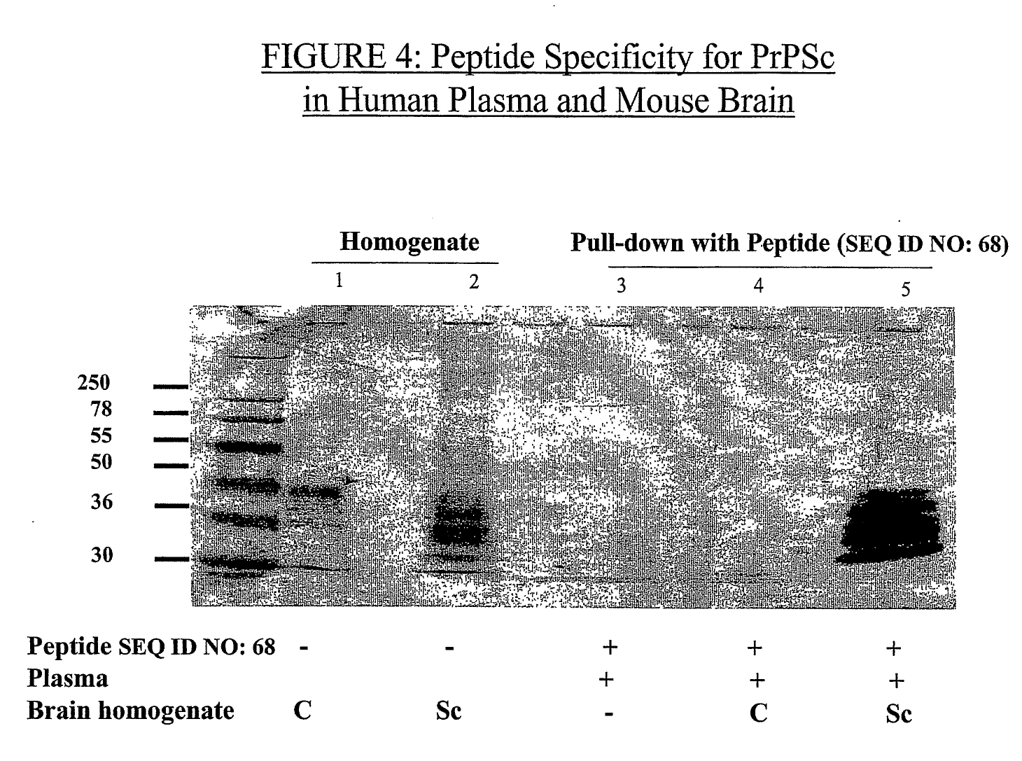

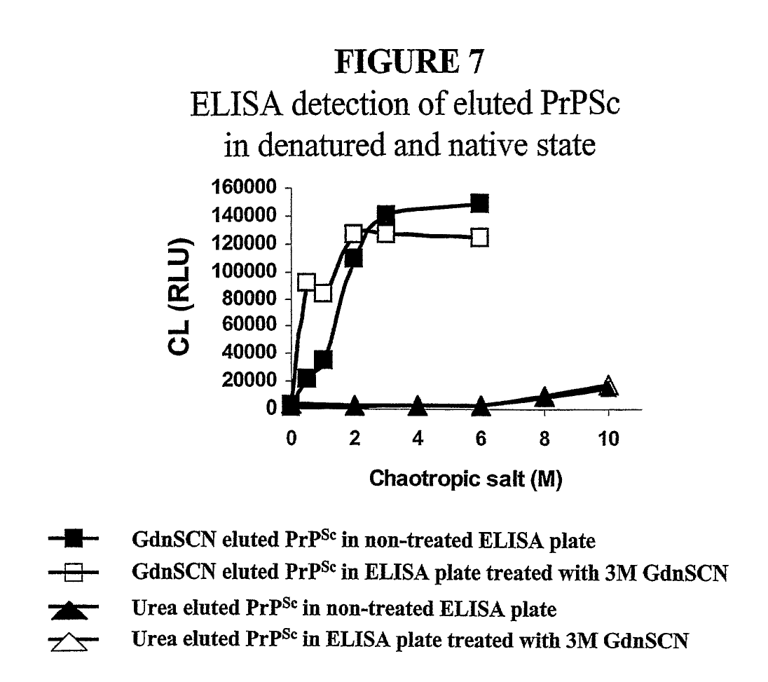

Results of exemplary ELISAs and Western blots are shown in Table 2 (O.D. values over blank controls (ranging from 0.172-0.259) were considered positive) and FIG. 7. In brief, proteinase K digestion of brain homogenates was not necessary in order to detect specific binding of the peptide reagents as described herein to bind to PrP^{Sc}. As shown in FIG. 4, in no case was binding observed to wild type brain homogenates, indicating that the peptide reagents were binding to PrP^{Sc} specifically. Furthermore, Western blotting analysis described above detected PrP^{Sc} at over four logs dilution while ELISA was at least 10× more sensitive than Western blotting.

TABLE 2

| | | | |
|---|---|---|---|
| Peptide reagent (biotin | | | |
| labeled on N- or | | Western | ELISA |
| C-terminal) | Seq Id: | Blot1 | A₄₀₅ₙₘ |

| | | | |
|---|---|---|---|
| ³CGG5WGQGGGTHNQWNKPSKPKTN | 35 | + | 0.687 |
| LKHV₃C | | | |
| ₃GGWGQGGGTHNQWNKPSKPKTNLKH | 36 | + | ND |
| GGWGQGGGTHNQWNKPSKPKTNLK | 37 | + | ND |
| HV₃ | | | |
| C₅GGWGQGGGTHNQWNKPSKPKTNL | 40 | + | ND |
| KHV₃C | | | |
| TPMIHFGNDWEDRYYRENMYR₄ | 44 | − | ND |
| ₄PMIHFGNDWEDRYYRENMYR₅C | 76 | − | ND |
| ₅C₄RPMIHFGNDWEDRYYRENMYR₄ | 46 | + | ND |
| C2 | | | |
| QWNKPSKPKTN₄ | 50 | + | 0.932 |
| QWNKPSKPKTN | 14 | +++ | 0.775 |
| QWNKPSKPKTN4QWNKPSKPKTN | 51 | +++ | 0.923 |
| QWNKPSKPKTNLKHV₄ | 77 | ++ | 0.839 |
| GGWGQGGGTHNQWNKPSKPKTN | 53 | + | 0.254 |
| GGTHNQWNKPSKPKTN | 54 | + | 0.253 |
| ₄AGAAAAGAVVGGLGQYMLGSAM | 78 | insoluble | 0.259 |
| ₄AGAAAAGAVVGGLGG | 56 | insoluble | 0.313 |
| ₆AGAAAAGAVVGGLGGYMLGSAM | 57 | + | 0.901 |
| ₆AGAAAAGAVVGGLGG | 65 | ++ | 0.635 |
| ₄KKRPKPGGWNTGGSRYPGQGS | 66 | + | 0.533 |
| ₄KKRPKPGGWNTGG | 67 | ++ | 0.451 |
| ₄KKRPKPGG | 68 | +++ | 0.765 |
| PHGGGWGQPHGGSWGQPHGGSWGQ | 69 | − | 0.282 |
| PHGGGWGQPHGGSWGQ | 70 | − | 0.241 |
| PHGGGWGQ | 71 | − | 0.263 |
| ₄GPKRKGPK | 73 | + | 1.0621 |
| ₄WNKPSKPKT | 75 | − | 0.247 |
| ₄NKPSKPK | 79 | − | 0.24 |
| ₄KPSKPK | 80 | − | 0.225 |
| ₄KKRPKPGGGKKRPKPGG | 72 | + | 0.522 |
| ₄KKRPKPGGGQWNKPSKPKTN | 81 | + | 1.247 |
| KKKAGAAAAGAVVGGLGGYMLGSA | 82 | − | 0.340 |
| MDDD | | | |
| DDDAGAAAAGAVVGGLGGYMLGSA | 83 | − | 0.237 |
| M | | | |
| KKKAGAAAAGAVVGGLGGYMLGSA | 84 | + | 0.268 |
| MKKK | | | |
| ₄KKKKKKKK | 85 | +³ | 0.530 |
| DDDAGAAAAGAVVGGLGGYMLGSA | 86 | − | 0.227 |
| MDDD | | | |
| ₄NNKQSPWPTKK | 87 | − | 0.277 |
| DKDKGGVGALAGAAVAAGGDKDK | 88 | − | 0.282 |
| ₄QANKPSKPKTN | 89 | + | 0.245 |
| ₄QWNKASKPKTN | 90 | − | 0.283 |
| ₄QWNKPSKAKTN | 91 | − | 0.256 |
| ₄QWNAPSKPKTN | 92 | − | 0.230 |
| ₄QWNKPSAPKTN | 93 | − | 0.250 |
| ₄QWNKPSKPATN | 94 | − | 0.260 |
| ₄QWNKASKAKTN | 95 | − | 0.241 |
| ₄KKRAXPGG | 96 | + | 2.19 |
| ₄KKRPKAGG | 97 | + | 1.24 |
| ₄KKRAKAGG | 98 | + | 1.46 |

1: Visually evaluated relative signal intensity
2: cydized
3: GGGG residues added/inserted at indicated position
4: GGG residues added/inserted at indicated position
5: GG residues added/inserted at indicated position
6: KKK residues added/inserted at indicated position
ND = not determined

Alanine scanning was also performed to identify residues involved in binding. Results are shown in Table 3.

TABLE 3

| | | | |
|---|---|---|---|
| Peptide reagent (biotin | | | |
| label on N- or C- | SEQ ID | Western | ELISA |
| terminus) | NO | Blot | A₄₀₅ₙₘ |

| | | | | |
|---|---|---|---|---|
| QWNKPSKPKTN | 14 | +++ | 0.775 | / |
| QANKPSKPKTN | 89 | +++ | 0.245 | |
| QWNAPSKPKTN | 92 | + | 0.283 | |
| QWNKPSAPKTN | 93 | + | 0.256 | |
| QWNKPSKPATN | 94 | + | 0.230 | |
| QWNKASKPKTN | 99 | +/− | 0.250 | |
| QWNKPSKAKTN | 91 | + | 0.260 | |
| QWNKASKAKTN | 95 | − | 0.241 | |
| QWAKPSKPKTN | 100 | ND | 0.376 | |
| QWNKPAKPKTN | 101 | ND | 0.356 | |
| QWNKPSKPKAN | 102 | ND | 0.234 | |
| QWNKPSKPKTA | 103 | ND | 0.262 | |
| KKRPKPGG | 68 | +++ | 0.765 | |
| AKRPKPGG | 104 | + | 0.273 | |
| KARPKPGG | 105 | + | 0.256 | |
| KKAPKPGG | 106 | + | 0.268 | |
| KKRPAPGG | 107 | + | 0.578 | |
| KKRAKPGG | 96 | ++ | 2.19 | |
| KKRPKAGG | 97 | ++ | 1.24 | |
| KKAPKAGG | 108 | + | 1.46 | |

In addition, as shown in Table 4, binding to PrP^{Sc} by the peptide reagents having SEQ ID NO:14, SEQ ID NO: 67 and SEQ ID NO:68 was further enhanced by substitutions at the proline residues by a number of N-substituted glycines (peptoids).

TABLE 4

| | |
|---|---|
| Western | ELISA |
| Blot | A₄₀₅ₙₘ |

*in (GGG)¹QWNKPSK*KTN (SEQ ID NO:14)

| | | |
|---|---|---|
| Proline | +++ | 0.775 |
| N-(S)-(1-phenylethyl)glycine | ++ | 0.865 |
| (peptoid as circled in FIG. | | |
| 3A) | | |
| (SEQ ID NO:109) | | |
| N-(4-hyclroxyphenyl)glycine | − | 0.934 |
| (peptoid as circled in FIG. | | |
| 3B) | | |
| (SEQ ID NO:110) | | |
| N-(cyclopropylmethyl)glycine | | |
| (peptoid as circled in FIG. | +++++ | 1.141 |
| 3C) | | |
| (SEQ ID NO:111) | | |
| N-(isopropyl)glycine | ND | 0.974 |
| (peptoid as circled in FIG. | | |
| 3D) | | |
| (SEQ ID NO:112) | | |
| N-(3,5-dimethoxybenzyl)glycine | +++ | 2.045 |
| (peptoid as circled in | | |
| FIG. 3E) | | |
| (SEQ ID NO:113) | | |
| N-butylglycine (peptoid as | ++++ | 0.776 |
| circled in FIG. 3F) | | |
| (SEQ ID NO:114) | | |

*in (GGG)¹KKRPK*GG (SEQ ID NO:14)

| | | |
|---|---|---|
| N-(cyclopropylmethyl)glycine | ND | 0.498 |
| (SEQ ID NO:115) | | |
| N-(isopropyl)glycine | ND | 1.57 |
| (SEQ ID NO:116) | | |
| N-(3,5-dimethoxybenzyl)glycine | ND | 0.823 |
| (SEQ ID NO:117) | | |
| N-butylglycine | ND | 0.619 |
| (SEQ ID NO:118) | | |

*in (GGG)¹KKRPK*GG (SEQ ID NO:68)

| | | |
|---|---|---|
| proline | ND | 0.765 |
| N-butylglycine | ND | 0.61 |
| (SEQ ID NO:119) | | |
| N-(3,5-dimethoxybenzyl) | ND | 0.631 |
| glycine | | |
| (SEQ ID NO:120) | | |
| N-(isopropyl)glycine | ND | 0.509 |
| (SEQ ID NO:121) | | |
| N-(cyclopropylmethyl)glycine | ND | 0.503 |
| (SEQ ID NO:122) | | |

*in (GGG)¹KKRPK*GGWNTGG (SEQ ID NO:67)

| | | |
|---|---|---|
| Proline | ND | 0.451 |
| N-butylglycine | ND | 0.503 |
| (SEQ ID NO:123) | | |
| N-(3,5-dimethoxybenzyl)glycine | ND | 0.464 |
| (SEQ ID NO: 124) | | |
| N-(isopropyl)glycine | ND | 0.555 |
| (SEQ ID NO:125) | | |
| N-(cyclopropylmethyl)glycine | ND | 0.344 |
| (SEQ ID NO:126) | | |

(GGG)¹QWNKX1SKX2KTN

| | | |
|---|---|---|
| N-(cyclopropylmethyl)glycine | ND | ND |
| at X1; N-(cyclopropylmethyl) | | |
| glycine at X2 | | |
| (SEQ ID NO:129) | | |
| N-(cyclopropyhiaethyl)glycine | ND | ND |
| at X1; N-(3,5-dimethoxybenzyl) | | |
| glycine at X2 | | |
| (SEQ ID NO:130) | | |
| N-(cyclopropylmethyl)glycine | ND | ND |
| at X1; N-butylglycine at | | |
| X2 | | |
| (SEQ ID NO:131) | | |
| N-(isopropyl)glycine | ND | ND |
| at X; N-(cyclopropylmethyl) | | |
| glycine at X2 | | |
| (SEQ ID NO:132) | | |
| N-(isopropyl)glycine | ND | ND |
| at X1; N-(3,5-dimethoxybenzyl) | | |
| glycine at X2 | | |
| (SEQ ID NO:257) | | |
| N-(isopropyl)glycine | ND | ND |
| at X1; N-butylglycine at X2 | | |
| (SEQ ID NO:258) | | |

*in (GGG)¹KKR*KPGGWNTGG (SEQ ID NO:67)

| | | |
|---|---|---|
| N-butylglycine | ND | ND |
| (SEQ ID NO:249) | | |
| N-(3,5-dimethoxybenzyl)glycine | ND | ND |
| (SEQ ID NO:250) | | |
| N-(isopropyl)glycine | ND | ND |
| (SEQ ID NO:251) | | |
| N-(cyclopropylmethyl)glycine | ND | ND |
| (SEQ ID NO:252) | | |

*in (GGG)1KKR*KPGG (SEQ ID NO:68)

| | | |
|---|---|---|
| N-butylglycine | ND | ND |
| (SEQ ID NO:253) | | |
| N-(3,5-dimethoxybenzyl)glycine | ND | ND |
| (SEQ ID NO:254) | | |
| N-(isopropyl)glycine | ND | ND |
| (SEQ ID NO:255) | | |
| N-(cyclopropylmethyl)glycine | ND | ND |
| (SEQ ID NO:256) | | |

¹The optional GGG linker was not present in the peptide reagents in the experiments shown in this table.

Furthermore, multimerization of PrP^{Sc}-binding peptide reagents also improved affinity for PrP^{Sc}. In particular, tandem repeats gave stronger signals (as measured by Western blotting) than single copies. Pre-derivatized MAP forms on beads increased binding in certain cases up to 2-fold. However, MAP forms caused precipitation of the peptide in solution. Linearly-linked peptides were also tested for their ability to enhance binding without causing precipitation.

### Example 3

### Isolation of Native or Denatured PrP^{Sc}

### A. Brain Homogenates

Brain homogenates were prepared essentially as described in Example 2 above. Briefly, one mouse brain was homogenized by douncing in ten volumes of a buffer containing Tris buffered saline (TBST) made up of Tris in 125 nM NaCl, triton X-100, and tween-20, pH 8.0. The homogenate was sonicated for 20 pulses to disperse aggregates and centrifuged at 500G for 15 minutes. The homogenate was then treated with proteinase K for 2 hours at 37° C. The proteinase K reaction was stopped with PMSF. Subsequent experiments showed the proteinase K treatment to be unnecessary.

### B. Binding to Peptide Reagents

For dissociation experiments, the homogenates were then bound to one of the following peptide reagents: QWNKPSKPKTN-biotin (SEQ ID NO:14); biotin-GGGKKRPKPGGWNTGG (SEQ ID NO:67); biotin-GGGKKRPKPGG (SEQ ID NO:68); biotin-GGGKKKKKKKK (SEQ ID NO:85); biotin-KKKAGAAAAGAVVGGKGGYMLGSAMKKK (SEQ ID NO:84); or the peptide reagent (containing a peptoid substitution) as depicted in SEQ ID NO:111. Binding of PrP^{Sc} in the samples to the peptide reagent was performed essentially as described in Example 2. Briefly, 400 μl of TBST buffer was mixed with 50 μl of the homogenate and 5 μl of a 10 mM peptide solution in a 1.5 mL tube. The tube was shaken for 1-2 hours at room temperature. Subsequently, 50 μl of magnetic streptavidin Dynabeads™ were added and the shaken for an hour at room temperature. The beads were washed 3 to 6 times in TBST to remove unbound sample and excess peptide reagent.

### C. Effect of pH on Binding

The effect of pH on binding of PrP^{Sc} was also tested with peptide reagents QWNKPSKPKTN-biotin (SEQ ID NO:14); biotin-GGGKKRPKPGG (SEQ ID NO:68); and biotin-GGGKKKKKKKK (SEQ ID NO:85). All three peptide reagents bound equally well to PrP^{Sc}. Experiments were carried out at pH 4, 5, 6, 7, 8 and 9 and results showed more binding at pH 6, 7, 8 or 9 than at pH 4 or 5. In particular, binding was worst at pH 4, steadily improving with increasing pH up to 8 and decreasing slightly at pH 9. Optimal binding was seen at pH 8.

### Example 4

### Dissociation of PrP^{Sc} from Peptide Reagents

The ability to dissociate (elute) PrP^{Sc} bound to peptide reagents described herein in native form was also tested using a variety of protocols.

### A. SODIUM CHLORIDE

The ability of NaCl to dissociate PrP^{Sc} bound to peptide reagents as described herein was evaluated. Following binding and washing as described above, the peptide reagent-PrP^{Sc} complexes were eluted with TBST buffer containing 1.0M or 1.2M NaCl for 20 minutes buffer was removed and the presence of PrP^{Sc} in the elutant was evaluated by Western Blot. In addition, the presence of any remaining peptide reagent-bound PrP^{Sc} was evaluated by Western blot by boiling the magnetic beads in SDS sample buffer for 5 minutes to strip PrP^{Sc} from the peptides.

Both 1.0 M and 1.2 M NaCl dissociated at least some PrP^{Sc} from the peptide reagent beads. PrP^{Sc} bound most tightly to SEQ ID NO:111 and little was dissociated with NaCl. However, PrP^{Sc} was recovered from peptide reagents biotin-GGGKKRPKPGG (SEQ ID NO:68), and biotin-GGGKKKKKKKK (SEQ ID NO:85) using NaCl dissociation, at about 40-50% recovery rates following 1.0 M NaCl elution. Furthermore, virtually all detectable PrP^{Sc} was dissociated from biotin-GGGKKRPKPGGWNTGG (SEQ ID NO:67) using 1.2 M NaCl.

Thus, non-denaturing concentrations of NaCl (e.g., 1.0 M to about 1.2M) can be used to dissociate PrP^{Sc} from peptide reagents as described herein.

### B. CHAOTROPIC SALTS

It has previously been shown that treatment of PrP^{Sc} with 1M to 2M GdnSCN for 24 hours does not significantly reduce PrP^{Sc} infectivity, while treatment with 3M GdnSCN for 1 hour reduced the majority of infectivity. (Prusiner et al. (1993) Proc. Nat'l Acad. Sci. USA 90:2793-2797). Similarly, treatment with 3M GdnSCN has been shown to denature PrP^{Sc} such that it can be detected with antibodies that recognize PrP^{C} and denatured PrP^{Sc} (e.g., D18). See, e.g., Peretz et al. (1997) J. Mol. Biol. 273(3):614-622; Williamson et al. (1998) J. Virol. 72(11):9413-9418. Thus, the use of chaotropic salts to dissociate PrP^{Sc} bound to peptide reagents under non-denaturing conditions was also evaluated as follows.

PrP^{Sc} was pulled down using SA-magnetic beads attached to biotinylated peptide reagents having with SEQ ID NO: 68 or SEQ ID NO:111 as described above. Following washing as described above (Example 3B), the peptide reagent-PrP^{Sc} complexes were mixed with TBST buffer containing 0, 0.5, 1, 3 or 6 M GdnSCN; 0, 0.5, 1, 3 or 6 M GdnHCl; or 0, 2, 4, 6, 8 or 10 M urea, all for 10-15 minutes at room temperature. The supernatant and beads were separated. The supernatant (elutant) was stored and the beads were washed and boiled in 2×SDS loading buffer

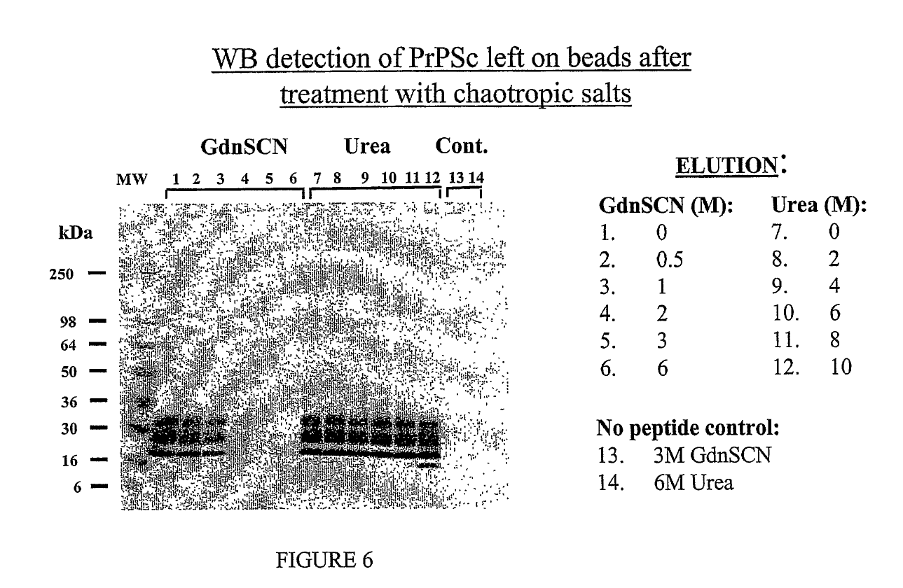

Western blot analysis was performed on the washed/boiled beads as described above to determine if any PrP^{Sc} remained bound to the beads. As shown in FIG. 6, 1M, 2M, 3M and 6M GdnSCN eluted some or most of the PrP^{Sc} bound to the peptide. However, urea, which is a weaker denaturant than GdnSCN did not dissociate PrP^{Sc} from the peptide-coated beads following 10-15 minute incubations, even at concentrations up to 10M.

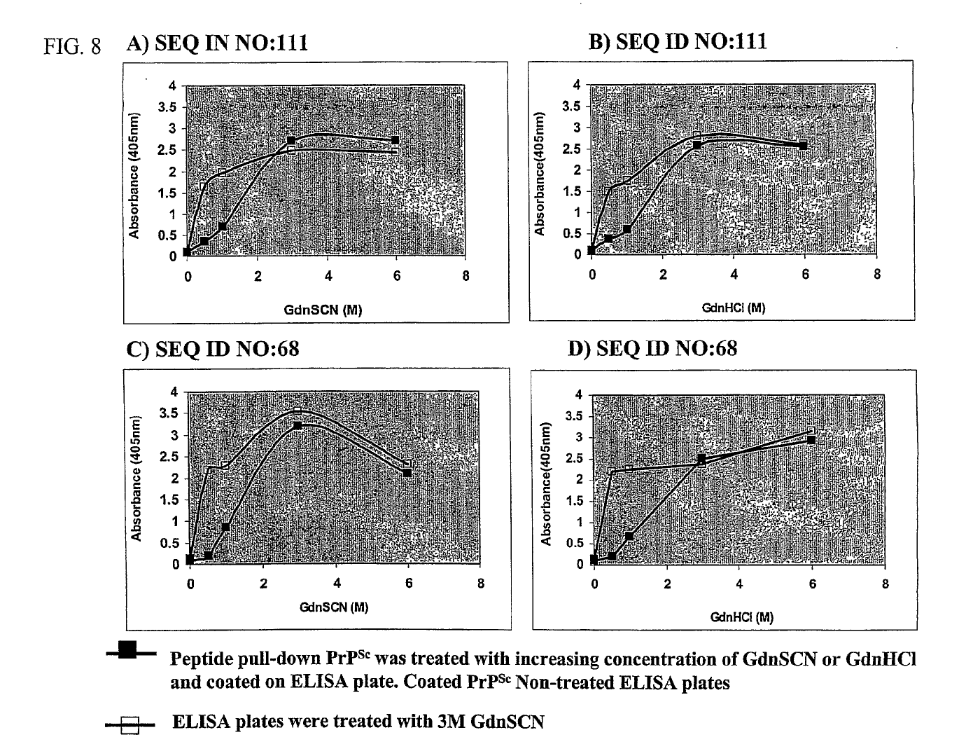

ELISA was also used to evaluate dissociation of PrP^{Sc} from peptide beads under denaturing and non-denaturing conditions. The supernatants prepared as described above were transferred to duplicate 96-well plates containing 50 μl/well of coating solution (0.1 NaCO₃, pH 8.8) and coated to the plates by incubation at 37° C. for 2 hours. To prepare the denatured samples, the coating buffer was aspirated from one set of the duplicate wells and 100 μl of 3M GdnSCN was added for 15 minutes at room temperature. The solution was then aspirated from all wells. The plate was washed 3× with 275 μl of washing buffer and blocked with 200 μl of 3% BSA in TBS for 1 hour at 37° C. and tested by ELISA. As shown in FIGS. 7 and 8, concentrations of GdnSCN or GdnHCl eluted PrP^{Sc} bound to the peptide reagent-coated beads in its native state. Urea did not dissociate PrP^{Sc} bound to the peptide reagent under the same experimental conditions.

In sum, these results demonstrate that PrP^{Sc} can be eluted from peptide reagents as described herein using 0.5M to about 2M guanidinium salts and, in addition, that PrP^{Sc} eluted with these concentrations of guanidinium salts are in their native (non-denatured) form. The results also demonstrate that PrP^{Sc} can be dissociated from peptide reagents described herein in a denatured form using from about 3M to about 6M guanidinium salts.

### C. CENTRIFUGATION

The ability of high-speed centrifugation to purify PrP^{Sc} was also evaluated. Brain homogenates containing PrP^{Sc} were prepared as described above. The homogenate was centrifuged at 500×G for 15 minutes and the supernatant collected and centrifuged for 16,000×G for 10 minutes. The supernatant was collected and treated with Proteinase K as described above for 2 hours at 37° C. and stopped with 0.1 M PMSF. Following Proteinase K digestion, the sample was centrifuged at 16,000×G and the supernatant collected and centrifuged at 20,000×G for 200 minutes. Supernatants and pellets from each step were analyzed by silver stained SDS gel and Western blotting as described herein

Characteristic banding patterns of PrP^{Sc} (triple band in Western blot) were seen in the PK-treated supernatant of both 16,000×G and 20,000×G supernatants, indicating that high-speed centrifugation can be used to purify PrP^{Sc}.

### D. CENTRIFUGATION AND NACL DISSOCIATION

The ability of high-speed centrifugation in combination with NaCl elution to dissociate PrP^{Sc} bound to peptide reagents as described herein was also evaluated.

PrP^{Sc}-containing samples were bound to peptide reagents as described above. Bound PrP^{Sc} was dissociated using 30 μl of 1.2 M NaCl and the beads boiled in SDS sample buffer to evaluate the presence of any remaining PrP^{Sc} bound to the peptide reagent. The NaCl eluted fraction was collected and centrifuged at 20,000×G for one hour, and the supernatant and pellet analyzed by silver stained SDS gel and Western blotting.

Results of these experiments showed that 1.2 M NaCl eluted all PrP^{Sc} bound to the peptide reagent (biotin-GGGKKRPKPGGWNTGG (SEQ ID NO:67)). Furthermore, no PrP^{Sc} was detected in the supernatant of the high-speed centrifugation, indicating that high-speed centrifugation concentrates all PrP^{Sc} into the pellet.

### E. PRECIPITATION

To evaluate whether PrP^{Sc} can be isolated by precipitation, the following experiments were conducted. Four 1 ml samples of mouse brain homogenate, previously digested with Proteinase K and centrifuged at 500×G and 16,000×G, were treated with a 10%, 20%, 30% or 40% saturated solution of ammonium sulfate. The samples were incubated for 1 hour at room temperature and centrifuged for 10 minutes at 10,000×G. The pellets were resuspended in TBST and sonicated. Supernatants and pellets (resuspended and sonicated) were analyzed by SDS gel (silver stained) and Western blotting.

PrP^{Sc} precipitated with all ammonium saturated solutions tested, with the best recovery obtained using 10% or 20% saturated solutions for ammonium sulfate for precipitating.

### F. COMBINATIONS

PrP^{Sc} is purified from mouse brain homogenates using a combination of centrifugation and dissociation (elution) with salt and/or chaotropic agents. Briefly, brain homogenates are prepared as described above and the homogenates centrifuged for 15 minutes at 500×G. The supernatant is collected and centrifuged for 10 minutes at 16,000×G. The supernatant is again collected and centrifuged for 200 minutes at 20,000×G and the pellet resuspended in TBST. The resuspended pellet is incubated with biotin-labeled peptide reagent as described herein that preferentially interacts with pathogenic prion forms (e.g., GGGKKRPKPGGWNTGG (SEQ ID NO:67)) for 1 hour. The PrP^{Sc} bound to the peptide reagent is then dissociated with 1.2 M NaCl or 0.5MGdn SCN (or GdnHCl).

The combination of centrifugation, and salt or chaotropic agent induced-dissociation from peptide reagents results in significantly purified native form PrP^{Sc}.

### G. CONCLUSION

Thus, the foregoing experiments demonstrate that peptide reagents described herein can be used to isolate PrP^{Sc} from complex biological medium without the use of Proteinase K. Furthermore, these experiments also demonstrate that PrP^{Sc} can be dissociated (eluted) from the peptide reagents in either a native or denatured conformation. The ability to isolate PrP^{Sc} in native conformation has important ramifications and applications, for example in screening for chemicals, proteins, antibodies or any other molecules that can bind PrP^{Sc}.

Although preferred embodiments of the subject invention have been described in some detail, it is understood that obvious variations can be made without departing from the spirit and the scope of the claims as set out below.

1. A method for isolating a pathogenic prion protein in the native conformation comprising the steps of: (a) contacting a sample containing a pathogenic prion protein with a peptide reagent that interacts preferentially with pathogenic prion protein, under conditions that allow the binding of the pathogenic prion protein, if present in said sample, to the peptide reagent to form a first complex; and (b) removing unbound sample materials.

2. A method for isolating a pathogenic prion protein in the native conformation comprising the steps of: (a) contacting a sample containing a pathogenic prion protein with a peptide reagent that interacts preferentially with pathogenic prion protein, under conditions that allow the binding of the pathogenic prion protein, if present in said sample, to the peptide reagent to form a first complex; (b) removing unbound sample materials; and (c) dissociating the pathogenic prion protein from the peptide reagent in non-denaturing conditions.

3. A method of isolating a pathogenic prion protein in the native conformation, the method comprising the steps of: (a) providing a solid support comprising a first peptide reagent that interacts preferentially with pathogenic prion protein; (b) contacting the solid support with a sample under conditions which allow pathogenic prions, when present in the sample, to bind to the first peptide reagent; (c) removing unbound sample materials; and (d) dissociating the pathogenic prions from the solid support using non-denaturing conditions.

4. The method of claim 1, wherein the peptide reagent comprises a peptide derived from a sequence selected from the group consisting of SEQ ID NO: 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, and 260.

5. The method of claim 4, wherein the peptide reagent comprises a peptide having a sequence selected from the group consisting of SEQ ID NO: 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, and 260.

6. The method of claim 4, wherein the peptide reagent comprises a peptide having a sequence selected from the group consisting of SEQ ID NOs: 66, 67, 68, 72, 81, 96, 97, 98, 107, 108, 119, 120, 121, 122, 123, 124, 125, 126, 127, 14, 35, 36, 37, 40, 50, 51, 77, 89, 100, 101, 109, 110, 111, 112, 113, 114, 115, 116, 117, 118, 128, 129, 130, 131, 132, 56, 57, 65, 82, 84, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 249, 250, 251, 252, 253, 254, 255, 256, 257, 258, 259, and 260.

7. The method of claim 4, wherein the peptide reagent comprises a peptide having a sequence selected from the group consisting of SEQ ID NOs: 66, 67, 68, 72, 81, 96, 97, 98, 107, 108, 119, 120, 121, 122, 123, 124, 125, 126, 127, 133, 134, 135 133, 134, 135, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 249, 250, 251, 252, 253, 254, 255, and 256.

8. The method of claim 4, wherein the peptide reagent comprises a peptide having a sequence selected from the group consisting of SEQ ID NOs: 14, 35, 36, 37, 40, 50, 51, 77, 89, 100, 101, 109, 110, 111, 112, 113, 114, 115, 116, 117, 118, 129, 130, 131, 132, 128, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 247, 257, 258, 259, and 260.

9. The method of claim 4, wherein the peptide reagent comprises a peptide having a sequence selected from the group consisting of SEQ ID NOs: 56, 57, 65, 82, 84 and 136.

10. The method of claim 4, wherein the peptide reagent comprises a peptide having a sequence selected from the group consisting of SEQ ID NOs: 67, 68 and 111.

11. The method of claim 1, further comprising the step of separating the pathogenic prions from the peptide reagent.

12. The method of claim 11, wherein the separation is by centrifugation, precipitation, or removal of the peptide by magnetic pull down.

13. The method of claim 3, wherein the dissociation step comprises contacting the first complex with a non-denaturing concentration of a salt or a chaotropic agent.

14. The method of claim 13, wherein the salt is NaCl or KCl.

15. The method of claim 14, wherein the salt is NaCl.

16. The method of claim 13, wherein the chaotropic agent is guanidium isothiocyanate (GdnSCN) or guanidinium hydrochloride (GdnHCl).

17. The method of claim 14, wherein the salt concentration is between about 0.5M and about 2M.

18. The method of claim 17, wherein the salt concentration is between about 1.0M and about 1.5M.

19. The method of claim 18, wherein the salt concentration is 1.0M, 1.1M, 1.2M, 1.3M, or 1.4M.

20. The method of claim 16, wherein the concentration of the chaotropic agent is between about 0.4M and about 2M.

21. The method of claim 20, wherein the concentration of the chaotropic agent is between about 0.4M and about 1.0M.

22. The method of claim 21, wherein the concentration of the chaotropic agent is 0.5M, 0.6M, 0.7M, 0.8M, 0.9M or 1.0M.

23. (canceled)
